(19) United States
(12) Patent Application Publication
(10) Pub. No.: US 2015/0004175 A1
(43) Pub. Date: Jan. 1, 2015

(54) Compositions and Methods for Reducing CTL Exhaustion

(71) Applicant: YALE UNIVERSITY, NEW HAVEN, CT (US)

(72) Inventors: Susan Kaech, Guildford, CT (US); Jonathan Chen, Barrington, IL (US)

(21) Appl. No.: 14/364,714

(22) PCT Filed: Dec. 13, 2012
(86) PCT No.: PCT/US12/69477

§ 371 Date: Jun. 12, 2014

Related U.S. Application Data
(60) Provisional application No. 61/570,033, filed on Dec. 13, 2011.

(51) Int. Cl. A61K 39/395; A61K 31/7088; A61K 38/02

(52) U.S. Cl. CPC A61K 39/3955; A61K 38/02; A61K 31/7088
USPC 424/158.1; 514/1.1; 514/3.7; 514/3.8; 514/4.3; 514/19.3; 514/44 R; 514/44 A

(57) ABSTRACT

The compositions and methods described herein are useful for diminishing CTL exhaustion in a subject in need thereof, during an immune response to a viral infection or during an immune response to cancer, thereby leading to a greater CTL response against the viral infection or cancer. The invention relates to compositions and methods for the therapeutic intervention of signaling through EP2 and EP4, by inhibiting at least one of EP2, EP4, PGE2, or combinations thereof. The invention also relates to compositions and methods for the therapeutic intervention of signaling through EP2 and EP4, in combination with the therapeutic intervention of signaling through PD-1, by inhibiting at least one of EP2, EP4, PGE2, or combinations thereof, in combination with inhibiting at least one of PD-1, PD-L1, PD-L2, and combinations thereof.

### CROSS-REFERENCE TO RELATED APPLICATIONS

This application claims the benefit of priority from U.S. Provisional Application Ser. No. 61/570,033, filed on Dec. 13, 2011, the entire disclosure of which is incorporated by reference herein as if set forth herein in its entirety.

### BACKGROUND OF THE INVENTION

Nearly 10% of the world's population is chronically infected with human immunodeficiency virus (HIV), hepatitis B virus (HBV) or hepatitis C virus (HCV), and together these viruses cause more than 4 million deaths annually. These viral infections are associated with prolonged periods of high titer viremia and in the case of HIV, depletion of the CD4 T cell pool. HBV and HCV are resolved in a minority of cases, but the majority of subjects acquire chronic infections that lead to long-term liver disease and cancer in a large number of cases. The majority of the world is chronically infected with several herpes viruses, including Epstein-Barr virus (EBV), cytomegalovirus (CMV), varicella zoster virus (VZV) or herpes simplex virus (HSV), and these can cause cancer and other serious medical complications. Unfortunately, for most of these viruses, with the exception of HBV and VZV, there are no protective vaccines and the antiviral treatments for HIV and HCV are extremely costly and not available globally. The discovery of therapeutic treatments to control or cure such chronic viral infections are in great need, and those capable of boosting the antigen-specific T cell response without excessive immunopathology offer the most clinical promise.

The establishment of chronic viral infection is complex and multi-factorial. Many viruses that establish chronic infection have evolved strategies to evade immune detection that directly contribute to virus persistence, but another equally important process is the active immune suppression that occurs by the host to regulate the anti-viral immune response to limit immunopathology during infection. This point becomes evident, especially for cytotoxic T lymphocytes (CTLs), which by nature cause collateral damage. For instance, during HCV, HBV, and HIV infection in humans and lymphocytic choriomeningitis virus Clone 13 (LCMV-CL13) infection in mice, several negative regulators of the anti-viral immunity have been identified to suppress CTLs and while some are T cell extrinsic, such as regulatory T cells (Tregs), TGF-β and IL-10, others are T cell intrinsic, such as the inhibitory receptors PD-1, TIM-3, CTLA-4, and LAG-3. Over time, the persistence of antigen can lead to physical depletion of certain epitope-specific populations of CTLs (such as the DbNP396-404- and KbGP34-41-specific CD8 T cells during LCMV-CL13 infection) or to an aberrant state of CTL function, commonly referred to as exhaustion, characterized by reduced proliferation, survival and effector function. In these settings, the ability of CD8 T cells to produce IL-2, TNFα and IFNγ and kill virus-infected cells can be suppressed in a manner that is directly proportional to viral load (Lechner et al., 2000, J Exp Med 191:1499-1512; Wherry et al., 2007, Immunity 27(4):670-84). Although CTL exhaustion contributes to viral persistence, it also prevents excessive, and potentially fatal, immunopathology and likely helps to sustain populations of virus-specific CTLs in the face of persisting antigen by diminishing virus-specific T cell deletion via activation induced cell death (AICD). Additionally, the presence of exhausted CTLs has been documented in several types of tumors and many similarities in gene and protein expression, such as increased PD-1 and TIM3, are shared between CTLs in the two disease settings.

Identifying the factors that induce CTL deletion and exhaustion during chronic infection is a critical area of investigation and only a handful of factors have been discovered. The activity of the inhibitory receptors and cytokines on CD8 T cells directly contribute to CTL dysfunction during chronic viral infection because blocking of PD1:PD-L1 interactions can profoundly augment CTL numbers, effector functions and improve viral control. Likewise, blockade of IL-10 and TGFβ signaling also enhances CTL responses during chronic infections. Therefore, interruption of these factors, in particular PD-1:PD-L1 signaling, has become a major focus of T cell-oriented therapies for chronic viral infection and cancer, and it is showing a high frequency of objective responses in patients. Given the exciting therapeutic promise of PD-1:PD-L1 blockade, finding additional inhibitory receptors or ligands, and more efficacious combinations of treatments therein, is of great clinical interest.

Thus, there is a need in the art to identify new therapeutic approaches for intervening in CTL exhaustion. The present invention addresses this unmet need in the art.

### SUMMARY

The invention relates to compositions and methods for the therapeutic inhibition of signaling through EP2 and EP4, by inhibiting at least one of EP2, EP4, PGE2, or combinations thereof. The invention also relates to compositions and methods for the therapeutic intervention of signaling through EP2 and EP4, in combination with the therapeutic intervention of signaling through PD-1, by inhibiting at least one of EP2, EP4, PGE2, and combinations thereof, in combination with inhibiting at least one of PD-1, PD-L1, PD-L2, and combinations thereof.

In one embodiment, the invention is a method of treating CTL exhaustion in a subject in need thereof, the method comprising administering a therapeutically effective amount of at least one inhibitor to the subject, wherein after the at least one inhibitor is administered to the subject, the CTL exhaustion is reduced. In another embodiment, the invention is a method of preventing CTL exhaustion in a subject in need thereof, the method comprising administering a therapeutically effective amount of at least one inhibitor to the subject, wherein after the at least one inhibitor is administered to the subject, the CTL exhaustion is prevented. In some embodiments, the at least one inhibitor diminishes the level of at least one of EP2, EP4, and PGE2. In other embodiments, the at least one inhibitor diminishes the level of at least one of PD-1, PD-L1, and PD-L2. In various embodiments, the at least one inhibitor is at least one selected from the group consisting of a chemical compound, a protein, a peptide, a peptidomemetic, an antibody, a ribozyme, a small molecule chemical compound, and an antisense nucleic acid molecule. In some embodiments, the at least one inhibitor is at least one selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In various embodiments the subject has a viral infection or cancer. In a particular embodiment, the subject is a human.

In one embodiment, the invention is a method of treating CTL exhaustion in a subject in need thereof, the method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the CTL exhaustion is reduced. In another embodiment, the invention is a method of preventing CTL exhaustion in a subject in need thereof, the method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the CTL exhaustion is prevented. In various embodiments, the at least two inhibitor are selected from the group consisting of a chemical compound, a protein, a peptide, a peptidomemetic, an antibody, a ribozyme, a small molecule chemical compound, and an antisense nucleic acid molecule. In some embodiments, the at least two inhibitors are selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In various embodiments the subject has a viral infection or cancer. In a particular embodiment, the subject is a human.

In one embodiment, the invention is a method of treating a viral infection in a subject in need thereof, the method comprising administering a therapeutically effective amount of at least one inhibitor to the subject, wherein after the at least one inhibitor is administered to the subject, the viral infection is treated. In some embodiments, the at least one inhibitor diminishes the level of at least one of EP2, EP4, and PGE2. In other embodiments, the at least one inhibitor diminishes the level of at least one of PD-1, PD-L1, and PD-L2. In various embodiments, the at least one inhibitor is at least one selected from the group consisting of a chemical compound, a protein, a peptide, a peptidomemetic, an antibody, a ribozyme, a small molecule chemical compound, and an antisense nucleic acid molecule. In some embodiments, the at least one inhibitor is at least one selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In some embodiments the viral infection is a chronic viral infection. In various embodiments, the viral infection is at least one of human immunodeficiency virus (HIV), hepatitis B virus (HBV), hepatitis C virus (HCV), Epstein-Barr virus (EBV), cytomegalovirus (CMV), varicella zoster virus (VZV) and herpes simplex virus (HSV). In a particular embodiment, the subject is a human.

In another embodiment, the invention is a method of treating cancer in a subject in need thereof, the method comprising administering a therapeutically effective amount of at least one inhibitor to the subject, wherein after the at least one inhibitor is administered to the subject, the cancer is treated. In some embodiments, the at least one inhibitor diminishes the level of at least one of EP2, EP4, and PGE2. In other embodiments, the at least one inhibitor diminishes the level of at least one of PD-1, PD-L1, and PD-L2. In various embodiments, the at least one inhibitor is at least one selected from the group consisting of a chemical compound, a protein, a peptide, a peptidomemetic, an antibody, a ribozyme, a small molecule chemical compound, and an antisense nucleic acid molecule. In some embodiments, the at least one inhibitor is at least one selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In a particular embodiment, the subject is a human.

In one embodiment, the invention is a method of treating a viral infection in a subject in need thereof, the method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the viral infection is treated. In various embodiments, the at least two inhibitor are selected from the group consisting of a chemical compound, a protein, a peptide, a peptidomemetic, an antibody, a ribozyme, a small molecule chemical compound, and an antisense nucleic acid molecule. In some embodiments, the at least two inhibitors are selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In some embodiments the viral infection is a chronic viral infection. In various embodiments, the viral infection is at least one of human immunodeficiency virus (HIV), hepatitis B virus (HBV), hepatitis C virus (HCV), Epstein-Barr virus (EBV), cytomegalovirus (CMV), varicella zoster virus (VZV) and herpes simplex virus (HSV). In a particular embodiment, the subject is a human.

In another embodiment, the invention is a method of treating cancer in a subject in need thereof, the method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the cancer is treated. In various embodiments, the at least two inhibitor are selected from the group consisting of a chemical compound, a protein, a peptide, a peptidomemetic, an antibody, a ribozyme, a small molecule chemical compound, and an antisense nucleic acid molecule. In some embodiments, the at least two inhibitors are selected from the group consisting of AH6809, L161,982, PF-04418948, CJ-23423, an anti-EP2 antibody, and anti-EP4 antibody, an anti-PGE2 antibody, anti-PD-L1 antibody, anti-PDL-1 antibody and an anti-PDL-2 antibody. In a particular embodiment, the subject is a human.

In one embodiment, the invention is a method of identifying a test compound as an inhibitor of a gene, or gene product, that is associated with CTL exhaustion, the method including the steps of measuring at least one parameter of a gene, or gene product, that is associated with CTL exhaustion in the absence of the test compound, measuring the at least one parameter of a gene, or gene product, that is associated with CTL exhaustion in the presence of the test compound, comparing the measurement of the at least one parameter of a gene, or gene product, that is associated with CTL exhaustion in the presence of the test compound with the measurement in the absence of the test compound, identifying the test compound as an inhibitor of the gene, or gene product, that is associated with CTL exhaustion when the measurement of the parameter in the presence of the test compound is different than the measurement of the parameter in the absence of the test compound. In various embodiments, the test compound is at least one selected from the group consisting of: a chemical compound, a polypeptide, a peptide, a peptidomemetic, an antibody, a nucleic acid, an antisense nucleic acid, an shRNA, a ribozyme, and a small molecule chemical compound. In various embodiments, the gene or gene product associated with CTL exhaustion is at least one of the group consisting of EP2, EP4, PGE2, PDL-1, PDL-2, and PD-1. In one embodiment, the invention is an inhibitor. In another embodiment, the invention is a composition comprising an inhibitor.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a series of images of a fluorescence-activated cell sorting (FACS) analysis of the expression of LAMP 1, INFγ, IL-2, and TNFα in cells isolated from wt and EP2/4 double knockout (DKO) mice.

FIG. 2 depicts the results of experiments demonstrating that PGE2 receptor expression is increased on activated CD8+ T cells during lymphocytic choriomeningitis virus (LCMV) infection.

FIG. 3 depicts a schematic of an experimental protocol for assessing whether PGE2 promotes CD8+ T cell exhaustion.

FIG. 4 depicts the results of experiments demonstrating that the reduction of PGE2 signaling increases the number of virus-specific CD8+ T cells during LCMV infection. The absence of PGE2 signaling improves the CD8+ T cell response to chronic LCMV infection. Wt, EP2/4 DKO, or mPGES1 KO mice were infected with LCMV CL13 and were sacrificed at day 21 post-infection. Representative FACS plots for H-2 Db GP33 tetramer staining of CD8+ T cells and average frequencies and numbers of H-2 Db NP396, GP33, and GP276 tetramer-positive splenocytes are shown.

FIG. 5 depicts the results of experiments assessing the production of INFγ, TNFα, and IL-2 by CD8+ T cells stimulated with the H-2D^{b}-binding LCMV peptides GP276, GP33 and NP396. The absence of PGE2 signaling improves the CD8+ T cell cytokine response at day 21 of chronic LCMV infection

FIG. 6 depicts the results of experiments demonstrating that the concurrent reduction of PD-1 signaling and PGE2 signaling synergistically increases the number of virus-specific CD8+ T cells at day 42 of LCMV infection. GP276, GP33 and NP396 are H-2D^{b}-binding LCMV peptides.

FIG. 7 depicts the results of experiments demonstrating that the absence of PGE2 signaling improves CD8+ T cell cytokine responses at day 42 of chronic LCMV infection. Representative FACS plots from NP396 peptide stimulation and compiled data for NP396, GP33, or GP276 peptide stimulated CD8+ T cell production of IFNγ, TNFα, and IL-2 are shown.

FIG. 8 depicts the results of experiments demonstrating that the concurrent inhibition of PD-1 signaling and PGE2 signaling synergistically lowered serum viral (LCMV) titer.

FIG. 9, comprising FIGS. 9A-9E, depicts the results of experiments demonstrating that the inhibition of PD-1 signaling and PGE2 signaling augments CD8+ T cell proliferation and improves CD8+ T cell survival. EP2/EP4 DKO antigen-specific CD8+ splenic T cells do not appear to have a proliferative advantage, but may be less prone to apoptosis. The BRDU experiments included the addition of BRDU in the drinking water for the last week before sacrifice. Representative histograms for the NP396 tetramer-positive population (FIG. 9A) and compiled averages for all mice (FIG. 9B) are shown. Cells were stained for active caspases with CaspGLOW to detect apoptosis. 7-AAD positive (dead) cells were excluded from the gate because these cells in this assay tended to non-specifically bind tetramer. Representative CaspGLOW FACS plots of the NP396 tetramer-positive population (FIG. 9C) and compiled averages for all mice (FIG. 9D) are shown. MFIs of BIM staining for all mice were averaged for each of the indicated tetramers (FIG. 9E). The data are representative of two (three for BIM) experiments with at least three mice per group.

FIG. 10 depicts the results of experiments demonstrating that the concurrent inhibition of PD-1 signaling and EP2/EP4 signaling increased the number of virus-specific CD8+ T cells and increased cytokine production by CD8+ T cells. Six week old female C57BL/6 mice were depleted of CD4+ T cells via administration of 200 μg GK1.5 antibody i.p. the day before and after LCMV CL13 infection (2×10⁶ PFU i.v.). One group of mice was treated with AH6809 (an EP2 inhibitor; 200 ug per dose, oral gavage) and L161,982 (an EP4 inhibitor; 200 ug per dose, i.p., CAS 147776-06-5) once daily beginning at day 28 post infection. This group and the “α-PD-L1” group received anti-PD-L1 antibody 200 μg i.p. every three days beginning at day 28 post infection. Mice were sacrificed at day 42 post infection. Antigen specific cells were counted using tetramer staining. Intracellular staining was performed after peptide stimulation in presence of brefeldin A. Data are compiled from 2 independent experiments of 3-4 mice per group.

FIG. 11 depicts the results of experiments demonstrating that the concurrent inhibition of PD-1 signaling and EP2/EP4 signaling increased the number of virus-specific CD8+ T cells and increased cytokine production by CD8+ T cells. Six week old female C57BL/6 mice were depleted of CD4+ T cells via administration of 200 μg GK1.5 antibody i.p. the day before and after LCMV CL13 infection (2×10⁶ PFU i.v.). One group of mice was treated with PF-04418948 (an EP2 inhibitor; 200 ug per dose, oral gavage) and CJ-23423 (an EP4 inhibitor; 200 ug per dose, i.p.) once daily beginning at day 2 post infection. These mice were treated twice daily with these compounds at indicated doses from day 28-42 post infection. This group and the “α-PD-L1” group received anti-PD-L1 antibody 200 μg i.p. every three days beginning at day 28 post infection. Mice were sacrificed at day 42 post infection. Antigen specific cells were counted using tetramer staining. Intracellular staining was performed after peptide stimulation in presence of brefeldin A. Data are from a single experiment of four mice per group (except only two mice in untreated control group).

FIG. 12, comprising FIGS. 12A-12D, depicts the results of experiments demonstrating that PGE2 signaling directly suppresses CD8 T cell function via the receptors EP2 and EP4 during LCMV infection. (A) Ptger2 (EP2) and Ptger4 (EP4) mRNA message was measured by qPCR using CD8+ CD44+ splenocytes sorted by FACS from LCMV-Arm or LCMV-CL13 infected mice at the indicated day post-infection (d.p.i.). Naïve CD8+ CD44− splenocytes were sorted from uninfected mice. For each experiment, cells from each condition were sorted from the pooled splenocytes of 3-5 mice and data were collected as fold-change in expression relative to naïve. Data presented here are compiled from three independent experiments. Error bars depict S.E.M. Data were analyzed by ANOVA. For both plots, * denotes p<0.05. (B) Congenically labeled Wt or EP2/4 DKO P14+ TCR transgenic T splenocytes were adoptively transferred into Wt mice. These mice were then depleted of CD4 T cells, infected with LCMV-CL13, and sacrificed at day 8 p.i. Splenocyte T cell stimulations were performed with GP33-41 peptide with or without 40 μM PGE2. Representative FACS plots of the P14+ TCR Tg cell production of IFN-γ, TNF-α, and IL-2 are shown. (C) Compiled data from experiments described in (B) are presented for IFN-γ, TNF-α, and IL-2. The percentage of P14+ TCR Tg splenocytes that produce IFN-γ is shown. TNF-α and IL-2 production are depicted as the percent of P14+ TCR Tg IFN-γ+ cells that also produce these cytokines. Data are compiled from at least four experiments with at least four mice per group. (D) EP2/4 DKO and Wt P14+ T cells were peptide-stimulated as in (C), but in the presence of PGE2. For each mouse, the fraction of cells producing the indicated cytokine in the presence of PGE2 was compared against the fraction of cells producing that cytokine in the absence of PGE2. The fraction of retained function is shown. Data are compiled from at least three experiments with at least four mice per group. For (C) and (D), each dot represents one mouse and the line indicates the mean. Statistical analysis was performed by unpaired two-tailed t test.

FIG. 13, comprising FIGS. 13A-13E, depicts the results of experiments demonstrating that PGE2 deficient mice have greater numbers of antigen-specific cytokine producing cells. Wt, EP2/4 DKO, or mPGES1 KO mice were infected with LCMV-CL13 and were sacrificed at day 21 p.i. (A) Representative FACS plots for DbGP33 tetramer staining of splenic CD8 T cells isolated from the indicated mice are shown. Plots are gated on CD8 T cells. (B) Average frequencies and numbers of DbNP396, DbGP33, and DbGP276 tetramer-positive splenocytes are shown in the bar graphs. Error bars depict S.E.M. for each population. The total height of each bar graph represents the sum of these three tetramer populations averaged across all mice analyzed. Data analyses comparing these sum percentages and numbers were performed using ANOVA. Data presented are compiled from 5 experiments with 2-5 mice per group. (C) Representative FACS plots of cytokine production after GP33-41 peptide stimulation. The left column of dot plots shows the gating of IFN-γ+ cells from a CD8 T cell population for the indicated mice. The right column of dot plots shows production of TNF-α and IL-2 by the IFN-γ+ gated population. (D) Stacked bar graphs show compiled data for numbers of CD8 T cells producing the indicated cytokines in response to NP396-404, GP33-41, or GP276-286 peptide stimulation. Error bars depict S.E.M. for each population. Data analysis comparing the sums was performed using ANOVA. Data presented are compiled from 4 experiments with 2-5 mice per group. (E) Serum from mice was measured for viral content by Vero cell plaque assay. Viral load is presented here as plaque forming units (PFU) per mL of serum. Data presented are compiled from 2-3 experiments with 2-5 mice per group. Error bars depict the S.E.M. The dashed line depicts the approximate limit of detection of the assay for viral load in serum.

FIG. 14, comprising FIGS. 14A and 14B, depicts the results of experiments demonstrating that blockade of PD-L1 synergizes with PGE2 signaling deficiency to restore T cell hierarchy. EP2/4 DKO (or mPGES1 KO as indicated) and Wt mice were depleted of CD4 T cells and infected with LCMV-CL13. From day 28 to 42 p.i., mice were treated every third day with either anti-PDL1 or PBS. At day 42 p.i., mice were euthanized and organs were harvested. (A) Representative FACS plots for DbNP396 tetramer staining of CD8 T cells are shown in the left column. Average frequencies and numbers of DbNP396, DbGP33, and DbGP276 tetramer-positive splenocytes are shown for experiments examining EP2/4 DKO (middle column) and mPGES1 KO mice (right column). Error bars depict S.E.M. for each population. Data presented are compiled from 6 experiments with 2-4 mice per group. (B) Splenocytes were stimulated with NP396-404, GP33-41, or GP276-286 LCMV peptides. Representative dot plots of cytokine production in response to NP396-404 peptide stimulation are depicted in the left two columns. The left column shows the gating IFN-γ+ cells from a population of CD8 T cell population for the indicated mice. The right column shows production of TNF-α and IL-2 by the IFN-γ+ population. Compiled data for the number of cells producing IFN-γ, TNF-α, or IL-2 in response to NP396-404, GP33-41, or GP276-286 peptide stimulation for EP2/4 DKO (middle column) and mPGES 1 KO (right column) versus Wt are shown. Data analysis comparing the sums was performed using ANOVA. EP2/4 DKO data presented are compiled from 4 experiments with 2-4 mice per group. mPGES1 KO data presented are compiled from 2 experiments with 2-4 mice per group.

FIG. 15 depicts the results of experiments demonstrating that viral titers in mPGES1 KO mice mPGES1 KO and Wt mice depleted of CD4 T cells and infected with LCMV-CL13. From day 28 to 42 p.i., mice were treated every third day with either anti-PD-L1 or PBS. Serum and organs from mice were measured for viral load by Vero cell plaque assay. Dashed lines indicate approximate limit of detection of the assay for the indicated organ. Limit of detection for viral load in serum is approximately 50 pfu/mL. Data analysis was performed using ANOVA. Each dot represents one mouse. Serum data presented are compiled from 2 independent experiments with 2-4 mice per group.

FIG. 16, comprising FIGS. 16A and 16B, depicts the results of experiments demonstrating elevated PD-1 expression in antigen-specific CD8 T cells from EP2/4 DKO and mPGES 1 KO cannot explain synergistic expansion in NP396-specific population upon blockade of PD-L1. Tetramer-positive CD8 T cells from the experiments described in FIG. 14 were surface stained with antibody against PD-1. Data analysis was performed by ANOVA. For the EP2/4 DKO data, compiled MFI data from 4 experiments with 2-4 mice per group are shown. For the mPGES 1 KO data, compiled MFI data from 2 experiments with 2-4 mice per group are shown. Error bars depict the S.E.M.

FIG. 17, comprising FIGS. 17A-17C, depicts the results of experiments demonstrating that EP2/4 DKO antigen-specific CD8 splenic T cells have a survival advantage. Splenocytes from the experiments described in FIG. 14 were stained for BrdU, active caspases, and intracellular Bim (A) BrdU was added to the drinking water for the last week before sacrifice. Representative plots for BrdU in the NP396 tetramer-positive population are shown in the left column of histograms. Compiled averages for the percent of NP396-specific splenocytes positive for BrdU are shown in the bar graph below the histograms. (B) Splenocytes were stained for active caspases with CaspGLOW to detect apoptosis. Representative CaspGLOW plots of the NP396 tetramer-positive population are depicted in the middle column of dot plots. Compiled averages for the NP396 tetramer-positive populations are shown in the bar graphs below the representative plots. (C) Cells were stained for intracellular Bim. Representative plots for intracellular Bim in the NP396-specific population are shown in the right column of histograms. Compiled averages of MFIs for the Bim staining in the NP396 tetramer-positive population are shown in the bar graphs below. For the bar graphs in (A), (B), and (C), data were compiled from 2-3 experiments with three mice per group. Data analysis was performed using ANOVA. Error bars depict S.E.M.

FIG. 18, comprising FIGS. 18A and 18B, depicts the results of experiments demonstrating that antigen-specific CD8 T cells from PGE2 deficient mice are more functional on a per cell basis than those from Wt mice. (A) CD8 T cell splenocytes from the experiments described in FIG. 13 were surface stained for the indicated tetramers and PD-1. Data presented are compiled from 5 experiments with 2-5 mice per group. Plots show MFIs with error bars depicting S.E.M. (B) Stimulation and tetramer staining data from the experiments described in FIG. 13 are represented here to elucidate the fraction of virus-specific cells capable of producing the indicated cytokines. IFN-γ is displayed as the percentage of tetramer-positive CD8 T cells producing IFN-γ. TNF-α and IL-2 are shown as the percentage of virus-specific IFN-γ producing cells also producing TNF-α or IL-2 respectively. Data presented are compiled from 4 experiments with 2-5 mice per group. Error bars depict S.E.M. for each population. Data analysis comparing the sums was performed using ANOVA.

FIG. 19, comprising FIGS. 19A and 19B, depicts the results of experiments demonstrating that blockade of PD-L1 synergizes with PGE2 signaling deficiency to restore GP34-specific CD8 T cell population. CD8 T cell splenocytes from mice described in FIG. 14A were stained with KbGP34 tetramer. Average frequencies (A) and numbers (B) of GP34 tetramer-positive splenocytes are presented. Data presented are compiled from 4 experiments with 3 mice per group. Data analysis was performed using ANOVA. Error bars depict S.E.M. for each population.

FIG. 20 depicts the results of experiments demonstrating that IL-2 production by antigen-specific CD8 T cells from Wt and PGE2 signaling-deficient mice with or without anti-PD-L1 treatment. Stimulation and tetramer data from the experiments described in FIG. 14 are represented here to elucidate the fraction of virus-specific-cells capable of producing IL-2. IL-2 is presented as the percentage of virus-specific IFN-γ producing cells also making IL-2. Data presented are compiled from 4 experiments with 2-4 mice per group. Data analysis was performed using ANOVA. Error bars depict S.E.M. for each population.

FIG. 21 depicts the results of experiments demonstrating that EP2/4 DKO antigen-specific CD8 splenic T cells have a survival advantage. NP396-specific CD8 T cell data from FIG. 17 are presented alongside GP33-specific and GP276-specific data from the same experiments and analyzed in an identical manner. As in FIG. 17, data were compiled from 2-3 experiments with three mice per group. Data analysis was performed using ANOVA. Error bars depict S.E.M.

FIG. 22 depicts the results of experiments assessing PD-1 expression on antigen-specific CD8 T cells.

FIG. 23 depicts the results of experiments demonstrating a beneficial effect on the antiviral CTL response during chronic LCMV infection when mice are treated with EP2/4 antagonists in combination with anti-PDL1 at days 28-42 post-infection.

FIG. 24 depicts the results of experiments demonstrating a beneficial effect on the antiviral CTL response during chronic LCMV infection when mice are treated with EP2/4 antagonists at the beginning of infection, and then blocked later with anti-PDL1 mAb (i.e., beginning at day 28).

FIG. 25 depicts the results of experiments demonstrating a beneficial effect on the antiviral CTL response during chronic LCMV infection when mice are treated with anti-PGE2 antibody in combination with anti-PDL1 at days 28-42 post-infection.

FIG. 26 depicts the results of experiments demonstrating a beneficial effect on the antiviral CTL response during chronic LCMV infection when mice are treated with anti-PGE2 antibody at the beginning of infection, and then blocked later with anti-PDL1 mAb (i.e., beginning at day 28).

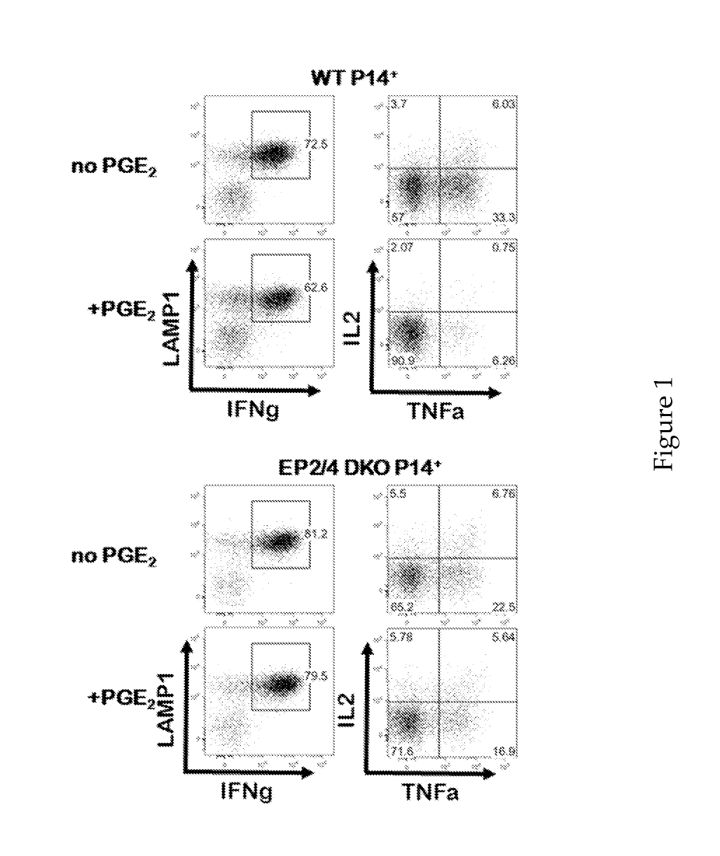

### DETAILED DESCRIPTION

The present invention relates to the discovery that the EP2 and EP4 are inhibitory receptors associated with CTL (e.g., CD8+ T cell) exhaustion. Both EP2 and EP4 are receptors of prostaglandin (e.g., PGE2). As described herein, subjects that are deficient in both EP2 and EP4, or deficient in PGE2 production, have CTLs that are more numerous and exhibit less exhaustion during chronic viral infection. Thus, the invention relates to compositions and methods for the therapeutic inhibition of signaling through EP2 and EP4, by inhibiting at least one of EP2, EP4, PGE2, or combinations thereof.

The invention also relates to the discovery that there is a synergistic interplay between PGE2 and PD1, another inhibitory receptor on CTLs. PD-1 is a receptor for PD-1 ligand 1 (PD-L1) and PD-1 ligand 2 (PD-L2). As described herein, inhibition of PD-1 signaling in subjects that are deficient for both EP2 and EP4, or deficient in PGE2 production, increases the number of CTLs and reverses exhaustion. Thus, the invention also relates to compositions and methods for the therapeutic intervention of signaling through EP2 and EP4, in combination with the therapeutic intervention of signaling through PD-1, by inhibiting at least one of EP2, EP4, PGE2, and combinations thereof, in combination with inhibiting at least one of PD-1, PD-L1, PD-L2, and combinations thereof.

In various embodiments, the compositions and methods for the therapeutic interventions described herein are useful for diminishing CTL exhaustion during a response to a viral infection or during a response to cancer, thereby leading to a greater CTL response against the viral infection or cancer.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the frequency or severity of a sign or symptom of a disease or disorder, experienced by a subject is reduced.

The term “cancer,” as used herein includes, but is not limited to, cancers of the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, melanoma, etc.), breast, genital system, (e.g., uterus, ovary, prostate, testis, etc.), urinary system (e.g, bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs or symptoms of pathology, for the purpose of diminishing or eliminating those signs or symptoms.

As used herein, “treating a disease or disorder” means reducing the frequency or severity of a sign or symptom of a disease or disorder experienced by a subject.

The phrase “biological sample” as used herein, is intended to include any sample comprising a cell, a tissue, or a bodily fluid in which expression of a nucleic acid or polypeptide can be detected. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

By the term “modulating,” as used herein, is meant mediating a detectable increase or decrease in the level or activity of a molecule, or in the response in a subject, compared with the level or activity of a molecule, or in the response in the subject, in the absence of a treatment or compound, and/or compared with the level or activity of an otherwise identical but untreated molecule or of a response in an otherwise identical but untreated subject. The term encompasses perturbing and/or affecting a native signal or response thereby mediating a beneficial therapeutic response in a subject, preferably, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to the discovery that EP2 and EP4 are inhibitory receptors associated with CTL (e.g., CD8+ T cell) exhaustion. Both EP2 and EP4 are receptors of prostaglandin (e.g., PGE2). As described herein, subjects that are deficient in both EP2 and EP4, or deficient in PGE2 production, have CTLs that are more numerous and exhibit less exhaustion during chronic viral infection. Thus, the invention relates to compositions and methods for the therapeutic inhibition of signaling through EP2 and EP4, by inhibiting at least one of EP2, EP4, PGE2, or combinations thereof.

The invention also relates to the discovery that there is a synergistic interplay between PGE2 and PD1, another inhibitory receptor on CTLs. PD-1 is a receptor for PD-1 ligand 1 (PD-L1) and PD-1 ligand 2 (PD-L2). As described herein, inhibition of PD-1 signaling in subjects that are deficient for both EP2 and EP4, or deficient in PGE2 production, synergistically increases the number of CTLs and reverses exhaustion. Thus, the invention also relates to compositions and methods for the therapeutic inhibition of signaling through EP2 and EP4, in combination with the therapeutic inhibition of signaling through PD-1, by inhibiting at least one of EP2, EP4, PGE2, and combinations thereof, in combination with inhibiting at least one of PD-1, PD-L1, PD-L2, and combinations thereof.

### Methods of Treatment

In various embodiments, the compositions and methods for the therapeutic inhibition described herein are useful for diminishing CTL exhaustion during a response to a viral infection or during a response to cancer, thereby leading to a greater CTL response against the viral infection or cancer.

In one embodiment, the present invention includes compositions and methods of reducing CTL exhaustion by diminishing the expression level, or activity level, of at least one of EP2, EP4, PGE2, or a combination thereof. In another embodiment, the invention includes compounds and methods for reducing CTL exhaustion by interfering with the interaction between PGE2 and at least one of its receptors (e.g., EP2 and EP4). In a further embodiment, the present invention includes compositions and methods of reducing CTL exhaustion by diminishing the expression level, or activity level, of at least one of EP2, EP4, PGE2, or a combination thereof, in combination with inhibiting at least one of PD-1, PD-L1, PD-L2, or a combination thereof. In a still further embodiment, the invention includes compounds and methods for reducing CTL exhaustion by interfering with the interaction between PGE2 and at least one of its receptors (e.g., EP2 and EP4), in combination with interfering with the interaction between PD-1 and at least one of its ligands (e.g., PD-L1 and PD-L2).

It would be understood by one skilled in the art, based upon the disclosure provided herein, that a decrease in the level of at least one of EP2, EP4, PGE2, PD-1, PD-L1, and PD-L2, encompasses a decrease of expression of at least one of EP2, EP4, PGE2, PD-1, PD-L1, and PD-L2. The skilled artisan would appreciate, once armed with the teachings of the present invention, that a decrease in the level of at least one of EP2, EP4, PGE2, PD-1, PD-L1, and PD-L2, includes a decrease in activity of at least one of EP2, EP4, PGE2, PD-1, PD-L1, and PD-L2. Thus, decreasing the level or activity of at least one of EP2, EP4, PGE2, PD-1, PD-L1, and PD-L2, includes, but is not limited to, decreasing transcription, translation, or both, of a nucleic acid encoding at least one of EP2, EP4, PGE2, PD-1, PD-L1, and PD-L2; and it also includes decreasing any activity of at least one of EP2, EP4, PGE2, PD-1, PD-L1, and PD-L2, as well. Additionally, the skilled artisan will appreciate, once armed with the teachings of the present invention, that the invention also includes compounds and methods for interfering with the interaction between PGE2 and at least one of its receptors (e.g., EP2 and EP4), as well as including compounds and methods for interfering with the interaction between PGE2 and at least one of its receptors (e.g., EP2 and EP4), in combination with interfering with the interaction between PD-1 and at least one of its ligands (e.g., PD-L1 and PD-L2).

A decrease in the level or activity of at least one of EP2, EP4, and PGE2, can be assessed using a wide variety of methods, including those disclosed herein, as well as methods well-known in the art or to be developed in the future. That is, the routineer would appreciate, based upon the disclosure provided herein, that decreasing the level or activity of at least one of EP2, EP4, and PGE2 can be readily assessed using methods that assess the level of a nucleic acid encoding EP2, EP4, or PGE2 (e.g., mRNA) and/or the level of EP2, EP4, or PGE2 protein present in a biological sample.

Similarly, a decrease in the level or activity of at least one of EP2, EP4, and PGE2, in combination with at least one of PD-1, PD-L1, and PD-L2, can be assessed using a wide variety of methods, including those disclosed herein, as well as methods well-known in the art or to be developed in the future. That is, the routineer would appreciate, based upon the disclosure provided herein, that decreasing the level or activity of at least one of EP2, EP4, and PGE2, in combination with at least one of PD-1, PD-L1, and PD-L2, can be readily assessed using methods that assess the level of a nucleic acid encoding EP2, EP4, PGE2, PD-1, PD-L1, or PD-L2, (e.g., mRNA) and/or the level of EP2, EP4, PGE2, PD-1, PD-L1, or PD-L2, protein present in a biological sample.

One skilled in the art, based upon the disclosure provided herein, would understand that the invention is useful in reducing CTL exhaustion in a subject who has, or is at risk of developing, a viral infection or cancer, whether or not the subject is also being treated with other medication or chemotherapy.

The inhibitor compositions of the invention useful for decreasing the level of at least one of EP2, EP4, PGE2, PD-1, PD-L1, and PD-L2, can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One of skill in the art would readily appreciate, based on the disclosure provided herein, that an inhibitor composition of the invention encompasses a chemical compound that decreases the level or activity of at least one of EP2, EP4, PGE2, PD-1, PD-L1, and PD-L2. Additionally, an inhibitor composition of the invention encompasses a chemically modified compound, and derivatives, as is well known to one of skill in the chemical arts.

In one embodiment, the inhibitor is an anti-PD-1 antibody. In another embodiment, the inhibitor is an anti-PD-L1 antibody. In another embodiment, the inhibitor is an anti-PD-L2 antibody. In another embodiment, the inhibitor is an anti-PGE2 antibody, such as, but not limited to 2B5. In another embodiment, the inhibitor is an anti-EP2 antibody. In another embodiment, the inhibitor is an anti-EP4 antibody.

In one embodiment, the inhibitor is a small molecule antagonist of PD-1. In another embodiment, the inhibitor is a small molecule antagonist of PD-L1. In another embodiment, the inhibitor is a small molecule antagonist of PD-L2. In another embodiment, the inhibitor is a small molecule antagonist of PGE2. In another embodiment, the inhibitor is a small molecule antagonist of EP2, such as but not limited to PF-04418948 and AH6809. In another embodiment, the inhibitor is a small molecule antagonist of EP4, such as but not limited to, CJ-23423 and L161982.

Further, one of skill in the art would, when equipped with this disclosure and the methods exemplified herein, appreciate that an inhibitor composition includes such inhibitors as discovered in the future, as can be identified by well-known criteria in the art of pharmacology, such as the physiological results of inhibition of at least one of EP2, EP4, PGE2, PD-1, PD-L1, and PD-L2 as described in detail herein and/or as known in the art. Therefore, the present invention is not limited in any way to any particular inhibitor as exemplified or disclosed herein; rather, the invention encompasses those inhibitors that would be understood by the routineer to be useful as are known in the art and as are discovered in the future.

Further methods of identifying and producing an inhibitor of the invention are well known to those of ordinary skill in the art, including, but not limited, obtaining an inhibitor from a naturally occurring source (i.e., Streptomyces sp., Pseudomonas sp., Stylotella aurantium). Alternatively, an inhibitor can be synthesized chemically. Further, the routineer would appreciate, based upon the teachings provided herein, that an inhibitor can be obtained from a recombinant organism. Compositions and methods for chemically synthesizing inhibitors and for obtaining them from natural sources are well known in the art and are described in the art.

One of skill in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of at least one of EP2, EP4, PGE2, PD-1, PD-L1, and PD-L2. (Sambrook et al., 2012, Molecular Cloning: A Laboratory Manual, Cold Spring Harbor Laboratory, New York; Ausubel et al., 1997, Current Protocols in Molecular Biology, John Wiley & Sons, New York).

One of skill in the art will realize that diminishing the amount or activity of a molecule that itself increases the amount or activity of at least one of EP2, EP4, and PGE2, in combination with at least one of PD-1, PD-L1, and PD-L2, can serve in the compositions and methods of the present invention to decrease the amount or activity of at least one of EP2, EP4, and PGE2, in combination with at least one of PD-1, PD-L1, and PD-L2.

Antisense oligonucleotides are DNA or RNA molecules that are complementary to some portion of an mRNA molecule. When present in a cell, antisense oligonucleotides hybridize to an existing mRNA molecule and inhibit translation into a gene product Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of an antisense oligonucleotide to diminish the level of at least one of EP2, EP4, PGE2, PD-1, PD-L1, and PD-L2; or to diminish the amount of a molecule that causes an increase in the amount or activity of at least one of EP2, EP4, PGE2, PD-1, PD-L1, and PD-L2, thereby decreasing the amount or activity of at least one of EP2, EP4, PGE2, PD-1, PD-L1, and PD-L2.

Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing EP2, EP4, PGE2, PD-1, PD-L1, and PD-L2, or of a gene expressing a protein that increases the level or activity of EP2, EP4, PGE2, PD-1, PD-L1, and PD-L2 can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One of skill in the art will appreciate that inhibitors of at least one of EP2, EP4, PGE2, PD-1, PD-L1, and PD-L2, can be administered singly or in any combination. Further, inhibitors of at least one of EP2, EP4, PGE2, PD-1, PD-L1, and PD-L2 can be administered singly or in any combination in a temporal sense, in that they may be administered simultaneously, before, and/or after each other. One of ordinary skill in the art will appreciate, based on the disclosure provided herein, that inhibitors of at least one of EP2, EP4, PGE2, PD-1, PD-L1, and PD-L2 can be used to reduce CTL exhaustion, and that an inhibitor can be used alone or in any combination with another inhibitor to effect a therapeutic result.

It will be appreciated by one of skill in the art, when armed with the present disclosure including the methods detailed herein, that the invention is not limited to treatment of CTL exhaustion that is already established. Particularly, the CTL exhaustion need not have manifested to the point of detriment to the subject; indeed, the CTL exhaustion need not be detected in a subject before a treatment is administered. That is, significant CTL exhaustion does not have to occur before the present invention may provide benefit. Therefore, the present invention includes a method for preventing CTL exhaustion in a subject, in that an inhibitor, as discussed previously elsewhere herein, can be administered to a subject prior to the onset of CTL exhaustion, thereby preventing the CTL exhaustion.

One of skill in the art, when armed with the disclosure herein, would appreciate that the prevention of CTL exhaustion encompasses administering to a subject an inhibitor as a preventative measure against CTL exhaustion. As more fully discussed elsewhere herein, methods of decreasing the level or activity of EP2, EP4, or PGE2, in combination with at least one of PD-1, PD-L1, and PD-L2, encompass a wide plethora of techniques for decreasing not only activity of at least one of EP2, EP4, PGE2, PD-1, PD-L1, and PD-L2, but also for decreasing expression of a nucleic acid encoding of at least one of EP2, EP4, PGE2, PD-1, PD-L1, and PD-L2.

Further, the compositions and methods described herein for treating or preventing CTL exhaustion in vivo in a subject in need thereof, can also be used in an ex vivo context. By way of nonlimiting examples, the compositions and methods described herein can be used to treat autologous cell products (e.g., stem cells, hematopoietic stem cells, hematopoietic progenitor cells, CTLs, etc.) ex vivo, before their transplantation into the subject.

Additionally, as disclosed elsewhere herein, one skilled in the art would understand, once armed with the teaching provided herein, that the present invention encompasses a method of preventing a wide variety of diseases, disorders and pathologies where a reduction in CTL exhaustion mediates, treats or prevents the disease, disorder or pathology. Methods for assessing whether a disease relates to increased levels of CTL exhaustion are known in the art. Further, the invention encompasses treatment or prevention of such diseases discovered in the future.

The invention encompasses administration of an inhibitor of at least one of EP2, EP4, PGE2, PD-1, PD-L1, and PD-L2 to practice the methods of the invention; the skilled artisan would understand, based on the disclosure provided herein, how to formulate and administer the appropriate inhibitor to a subject. Indeed, the successful administration of the inhibitor has been reduced to practice as exemplified herein. However, the present invention is not limited to any particular method of administration or treatment regimen.

### Methods of Identifying an Inhibitor

The invention also relates to methods of identifying compounds that inhibit CTL exhaustion. In various embodiments, the method of identifying of the invention identifies an inhibitor compound that inhibits the level or activity of a gene, or gene product, that is associated with CTL exhaustion. In one embodiment, the method of identifying of the invention identifies an inhibitor compound that diminishes the level or activity of a gene, or gene product, that is associated with CTL exhaustion. In particular embodiments, the gene, or gene product, that is associated with CTL exhaustion is at least one of EP2, EP4, PGE2, PD-1, PD-L1, and PD-L2. The invention further comprises the inhibitor of CTL exhaustion, as well as compositions comprising the inhibitor of CTL exhaustion, identified by the methods described herein.

In one embodiment, the invention comprises a method of identifying a test compound as an inhibitor of CTL exhaustion. Generally, the method of identifying a test compound as an inhibitor of CTL exhaustion includes comparing a parameter of CTL exhaustion in the presence of a test compound with a parameter of CTL exhaustion in the absence of the test compound. Thus, in some embodiments, the method includes the steps of: measuring at least one parameter of CTL exhaustion in the absence of the test compound; measuring the at least one parameter of CTL exhaustion in the presence of the test compound; and comparing the level of the at least one parameter of CTL exhaustion in the presence of the test compound with the level of the at least one parameter of CTL exhaustion in the absence of the test compound; and identifying the test compound as an inhibitor of CTL exhaustion when the level of the at least one parameter of CTL exhaustion in the presence of the test compound is different than level of the at least one parameter of CTL exhaustion in the absence of the test compound.

In another embodiment, the invention comprises a method of identifying a test compound as an inhibitor of a gene, or gene product, that is associated with CTL exhaustion. Generally, the method of identifying a test compound as an inhibitor of a gene, or gene product, that is associated with CTL exhaustion, includes comparing a parameter of a gene, or gene product, in the presence of a test compound with a parameter of the gene, or gene product, in the absence of the test compound. Thus, in some embodiments, the method includes the steps of: measuring at least one parameter of the gene, or gene product, in the absence of the test compound; measuring the at least one parameter of the gene, or gene product, in the presence of the test compound; and comparing the level of the at least one parameter of the gene, or gene product, in the presence of the test compound with the level of the at least one parameter of the gene, or gene product, in the absence of the test compound; and identifying the test compound as an inhibitor of the gene, or gene product, when the level of the at least one parameter of the gene, or gene product, in the presence of the test compound is different than level of the at least one parameter of the gene, or gene product, in the absence of the test compound. In various embodiments of the method, the measured parameter of the gene, or gene product, is at least one of: the level of expression, the level of transcription, the level of translation, the level of nucleic acid, the level of protein, or the level of activity (e.g., enzymatic, binding, etc.). In various embodiments, the gene, or gene product, that is associated with CTL exhaustion, is at least one of EP2, EP4, PGE2, PD-1, PD-L1, and PD-L2

Suitable test compounds include, but are not limited to, a chemical compound, a polypeptide, a peptide, a peptidomemetic, an antibody, a nucleic acid, an antisense nucleic acid, an shRNA, a ribozyme, and a small molecule chemical compound. Other methods, as well as variations of the methods disclosed herein, will be apparent from the description of this invention. In various embodiments, the test compound concentration in the identification assay can be fixed or varied. A single test compound, or a plurality of test compounds, can be tested at one time.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical that new chemical entities with useful properties are generated by identifying a chemical compound (called a “lead compound”) with some desirable property or activity, creating variants of the lead compound, and evaluating the property and activity of those variant compounds.

In one embodiment, high throughput screening methods involve providing a library containing a large number of test compounds potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Pharmaceutical Compositions

Compositions identified as potentially useful inhibitor compounds for treatment and/or prevention of CTL exhaustion, can be formulated and administered to a subject for treatment and/or prevention of CTL exhaustion, as now described.

The invention encompasses the preparation and use of pharmaceutical compositions comprising a composition useful for treatment of CTL exhaustion, disclosed herein as inhibitor of a gene, or gene product, that is associated with CTL exhaustion. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor may be combined and which, following the combination, can be used to administer the appropriate inhibitor thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate inhibitor thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

The materials and methods used in this Experimental Example are now described.

### Mice, Infections, Treatments, and Plaque Assays

Six week old female C57BL/6 mice were obtained from NCI (Frederick, Md.). EP2 KO mice (Kennedy et al., 1999, Nat. Med. 5:217-220) on the C57BL/6 line were a gift from Dr. Chuanming Hao. Genotyping for the wild-type (wt) allele used the primers 5′-CCGGGGTTCTGGGGAATC-3′ (SEQ ID NO: 1) and 5′-GTGCATGCGAATGAGGTTGAG-3′ (SEQ ID NO: 2). Genotyping for the mutant allele used the primers 5′-TTGCCAAGTTCTAATTCCATCAGA-3′ (SEQ ID NO: 3) and 5′-GTGCATGCGAATGAGGTTGAG-3′ (SEQ ID NO: 4). EP4-floxed mice (Schneider et al., Genesis 40:7-14) on the C57BL/6 line were also a gift from Dr. Chuanming Hao. Genotyping the EP4-floxed mice used the primers 5′GTTAGATGGGGGGAGGGGACAACT-3′ (SEQ ID NO: 5) and 5′TCTGTGAAGCGAGTCCTTAGGCT-3′ (SEQ ID NO: 6). The floxed gene produced a 334 bp band, while the wt allele produced a 243 bp band. mPGES1−/− mice (Trebino et al., Proc Natl Acad Sci USA 100:9044-9049) on the C57BL/6 line were a gift from Dr. Daniel Rosenberg. Genotyping for the mutant allele used the primers 5′-GGAAAACCTCCCGGACTTGGTTTTCAG-3′ (SEQ ID NO: 7) and 5′-ATCGCCTTCTATCGCCTTCTTGACGAG-3′ (SEQ ID NO: 8). While genotyping for the wt allele used the primers 5′-GGAAAACCTCCCGGACTTGGTTTTCAG-3′(SEQ ID NO: 9) and 5′-CAGTATTACAGGAGTGACCCAGATGTG-3′ (SEQ ID NO: 10). EP4-floxed mice were crossed to Granzyme B-cre mice (Jacob and Baltimore, Nature 399:593-597) and then to the EP2−/− mice to create EP2/4 DKO mice.

EP2/4 DKO mice were crossed to Thy1.1+ P14 TCR transgenic (tg) mice (Kaech and Ahmed, Nat. Immunol. 2:415-422). 5,000 CD8+ Thy1.1+ P14 TCR tg cells were transferred i.v. into wt C57BL/6 mice to create “P14 chimeric mice” (Joshi et al., 2007, Immunity 27:281-295). C57BL/6 mice used as controls for all of the knockouts were derived from wt littermates to the knockout strains. Importantly, these knockout mice were housed in the same room of the mouse facility as the knockout mice. Mice were infected with 2×10⁵ PFU of LCMV-Armstrong (i.p.) or 2×10⁶ PFU of LCMV-CL13 (i.v.) as described by Joshi (Joshi et al., 2007, Immunity 27:281-295). For indicated experiments, mice were depleted of CD4+ T cells by administration of anti-CD4 antibody (clone GK1.5 at dose of 200 μg intraperitoneally) the day before and after infection. All animal experiments were done with approved Institutional Animal Care and Use Committee protocols.

Two sets of EP2 and EP4 inhibitor compounds were used. The first set was AH6809 (EP2 blocking) (CAS 33458-93-4, Cayman Chemical, Ann Arbor, Mich.) and L161,982 (EP4 blocking) (CAS 147776-06-5, Cayman Chemical, Ann Arbor, Mich.). The second set was with PF-04418948 (EP2 blocking) (Pfizer, Groton, Conn.; see, for example, of Forselles, 2011, Br J. Pharmacol. 164:1847-56) and CJ-23423 (EP4 blocking) (Pfizer, Groton, Conn.). For both sets of EP2 inhibitor compounds, the inhibitor compound was administered by oral gavage 200 μg in 100 μL corn oil). For both sets of EP4 inhibitor compounds, the inhibitor compound was administered i.p. (200 μg in 100 μL PBS). Control animals were given gavages of 100 μL corn oil and i.p. injections of 100 μL PBS. In cases where anti-PD-L1 (Bio X Cell, West Lebanon, N.H.) was administered, mice were i.p. injected with 200 μg of antibody in 200 μL of PBS.

### Cell Isolation, Surface, and Intracellular Staining

BrdU (Sigma, St. Louis, Mo.) was administered at 1 mg/mL in the drinking water. Detection of BrdU was performed following the instructions on the staining kit (BD, San Jose, Calif.). CaspGLOW staining was performed according to provided instructions (eBioscience, San Diego, Calif.). Tetramer staining and T cell stimulation with LCMV peptide was performed as previously described (Fuller et al., J. Immunol. 172:4204-4214). Intracellular staining for BIM (Cell Signaling Technology, Boston, Mass.), Bcl2 (R&D Systems, Minneapolis, Minn.), and transcription factors (eBioscience, San Diego, Calif.) was performed using FoxP3 Fix/Perm Buffer (BioLegend, San Diego, Calif.) and Perm/Wash Buffer (BD, San Jose, Calif.). PGE2 (Cayman Chemical, Ann Arbor, Mich.) was administered at 40 μM at the time of peptide administration for the indicated in vitro stimulations. Flow cytometry was performed on a LSRII machine (BD, San Jose, Calif.). Sorting was performed on a FACS Aria (BD, San Jose, Calif.).

Gene Expression by qRT-PCR and PGE2 Measurements

For qRT-PCR, RNA was isolated from 500,000 sorted cells following the instructions provided with the Qiashredder and RNeasy kits (Qiagen, Valencia, Calif.). cDNA was then synthesized using SSRTII (Invitrogen, Carlsbad, Calif.). Real time was performed on a Stratagene Mx3000P, as previously described (Joshi et al., 2007, Immunity 27:281-295). Relative fold changes were calculated using L9 expression as a normalization constant.

PGE2 measurement was performed by ELISA (Cayman Chemical, Ann Arbor, Mich.). For PGE2 measurement, media was harvested from cultures 24 hours after they were set up. Media was spun to precipitate out cellular debris, and the resulting supernatant was stored at −80° C. until its use in the PGE2 ELISA. Viral plaque assays were performed following von Herrath's protocol (von Herrath and Whitton, 2003, Animal Models Using Lymphocytic Choriomeningitis Virus. Current Protocols in Immunology, Unit 19.10). Briefly, Vero cells were plated in 6 well plates. When the Vero monolayer was ˜60% confluent, a dilution of either serum or tissue homogenate sample was added to each well. The virus in each sample was then given one hour to infect the Vero monolayer. Thereafter, an agarose gel containing serum, 199 media, and Seakem agarose was placed over the monolayer. These 6 well plates were then incubated for four days in a CO₂ incubator. After this period, formaldehyde was added to the wells and the plates were allowed to sit for 2 hours. Then, the agarose gel was removed and a 0.1% solution of crystal violet was added to each well. After 2 hours, the plates were washed in water. Plaques were then counted and the viral load was back-calculated using the volume of sample added and its dilution.

The results of this Experimental Example are now described.

### PGE2 Receptor Expression During Viral Infection

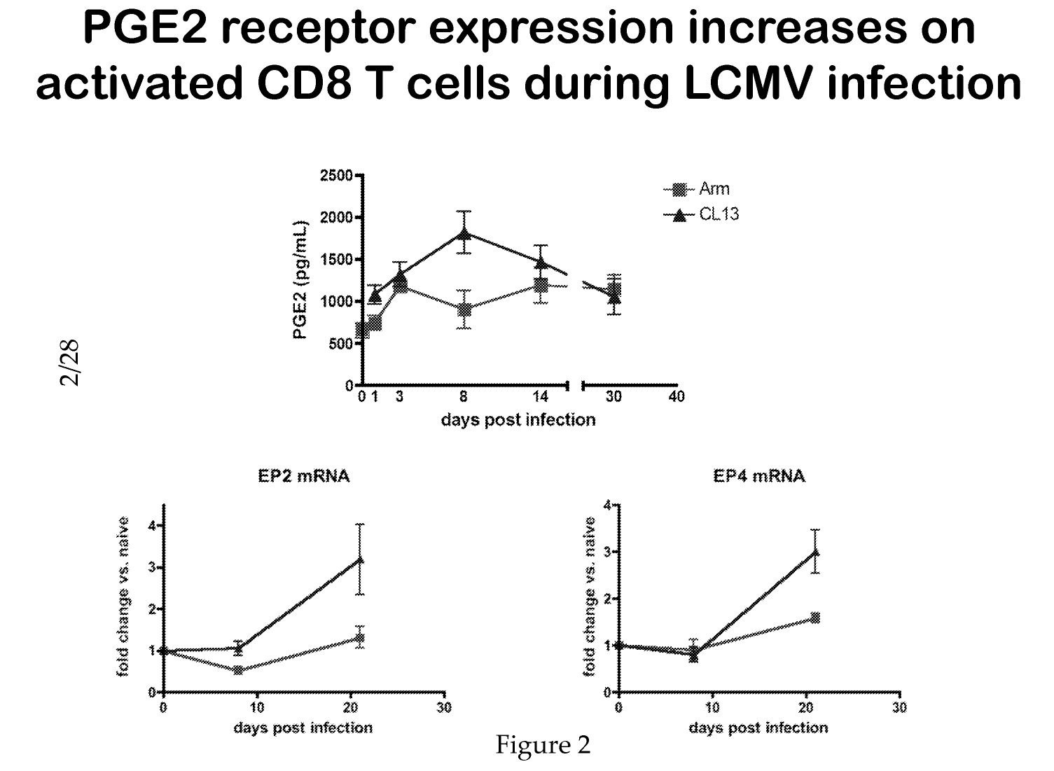

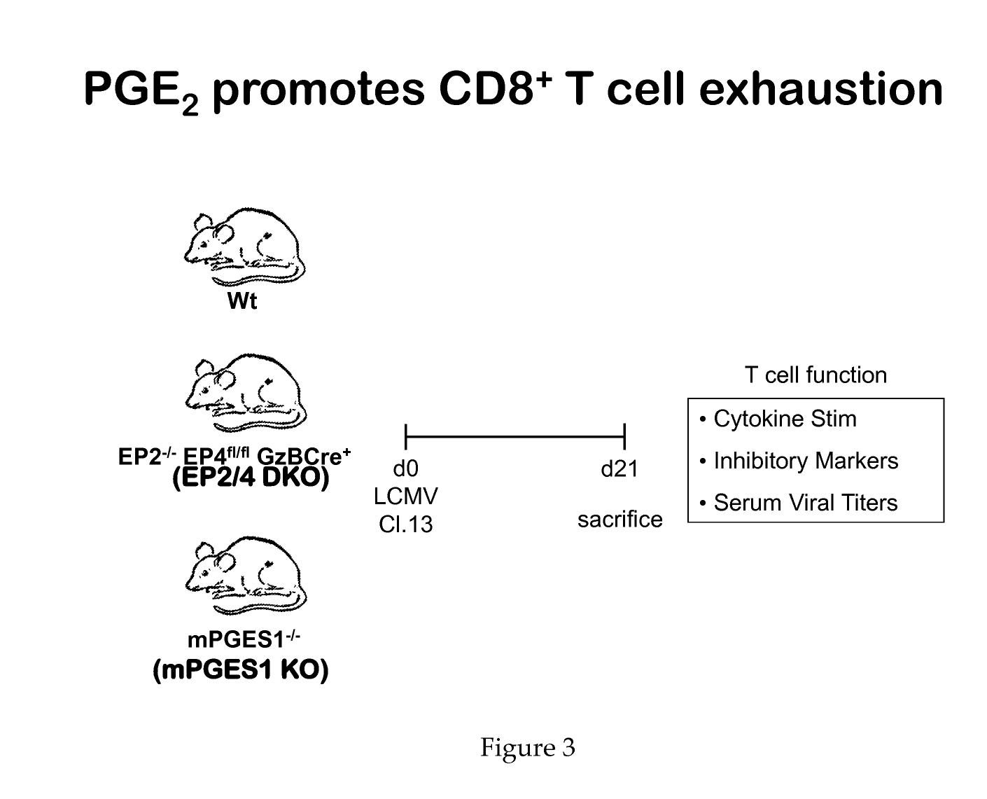

PGE2 receptor expression increases on activated CD8+ T cells during lymphocytic choriomeningitis virus (LCMV) infection. By Day 20 of LCMV infection, the expression of both EP2 and EP4 on CD8+ T cells was increased by about 3-fold (FIG. 2).

### Reduction in PGE2 Signaling Increases Virus-Specific CD8+ T Cells During Viral Infection

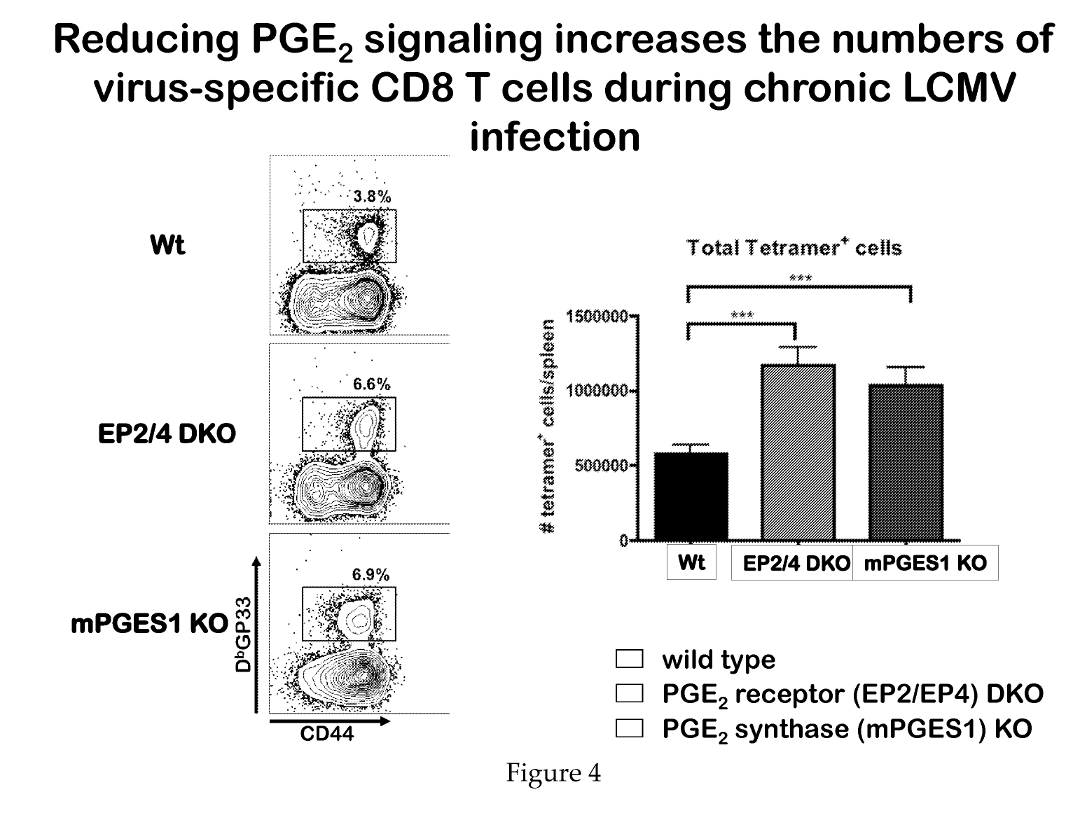

The reduction of PGE2 signaling increases the number of virus-specific CD8+ T cells during LCMV infection. The absence of PGE2 signaling improves the CD8+ T cell response to chronic LCMV infection. Wt, EP2/4 DKO, or mPGES1 (prostaglandin E synthase-1) KO mice were infected with LCMV CL13 and were sacrificed at day 21 post-infection. Representative FACS plots show the proportion of H-2 Db GP33 tetramer-positive CD8+ T cells is increased in EP2/EP4 DKO and mPGES1 KO mice, as compared with wt mice (FIG. 4). Additionally, the number of H-2 Db NP396, GP33, and GP276 tetramer positive splenocytes is increased in EP2/EP4 DKO and mPGES1 KO mice, as compared with wt mice (FIG. 4).

### Concurrent Inhibition of PD-1 and PGE2 Signaling

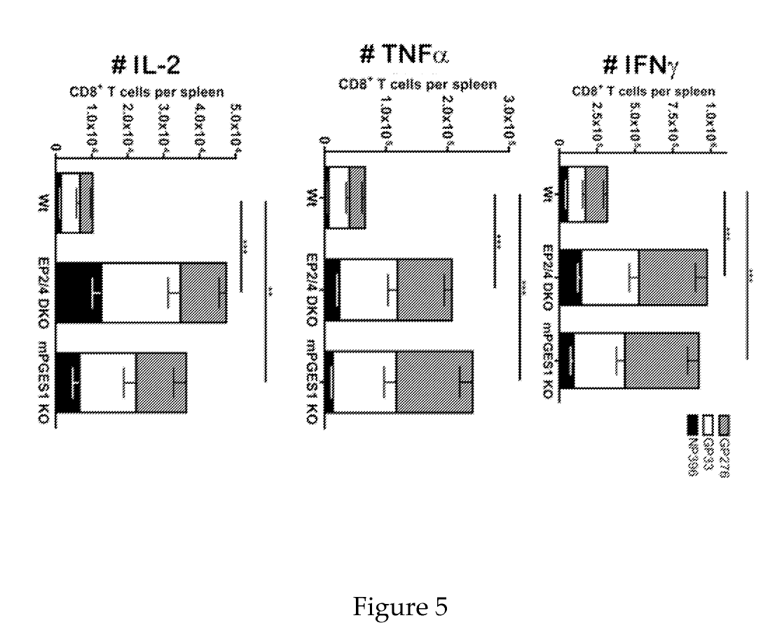

The absence of PGE2 signaling improves the CD8+ T cell cytokine response at day 21 of chronic LCMV infection. CD8+ T cells from EP2/EP4 DKO and mPGES1 KO mice produced more INFγ, TNFα, and IL-2 when stimulated with the H-2D^{b}-binding LCMV peptides GP276, GP33 and NP396, as compared with wt mice (FIG. 5).

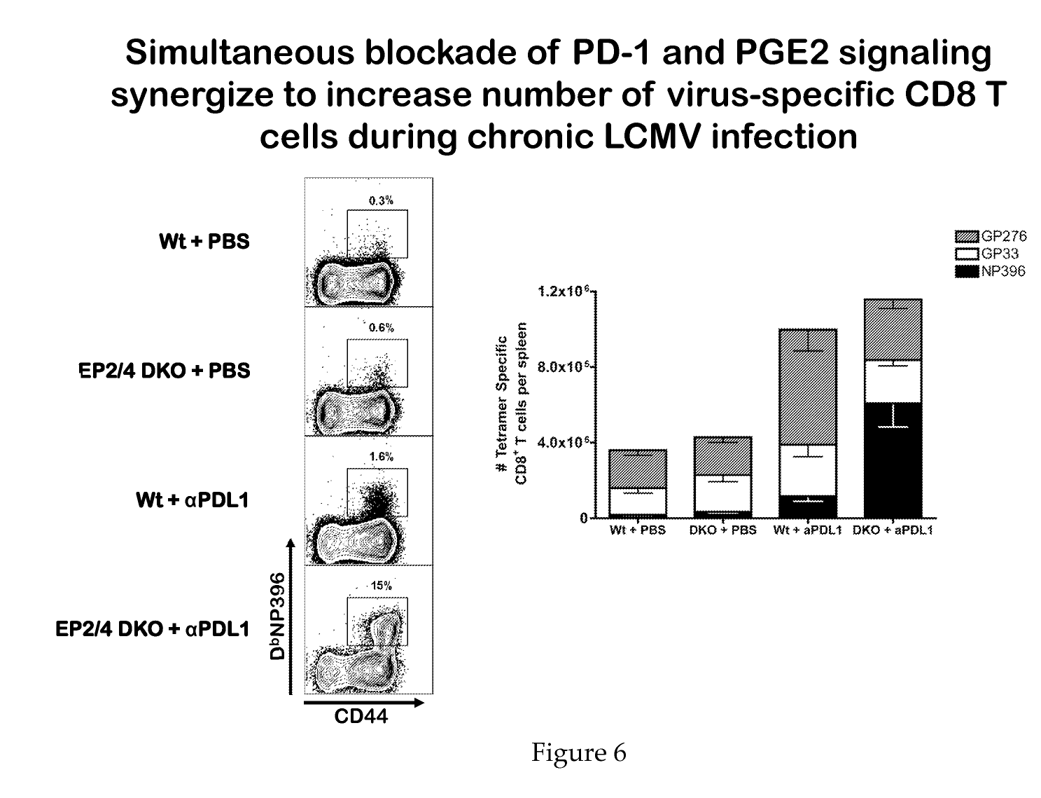

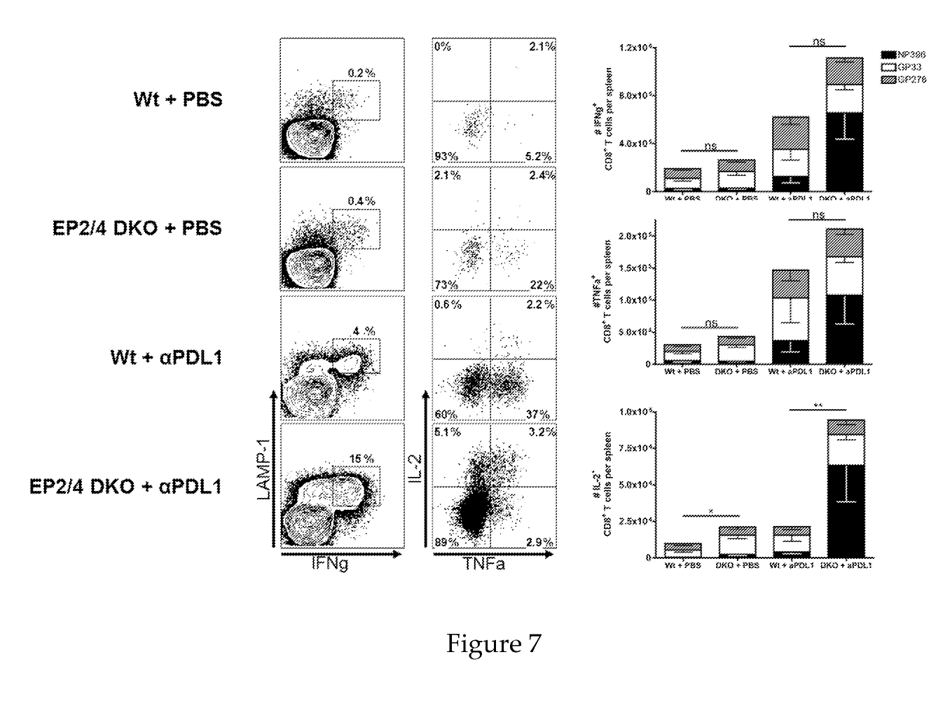

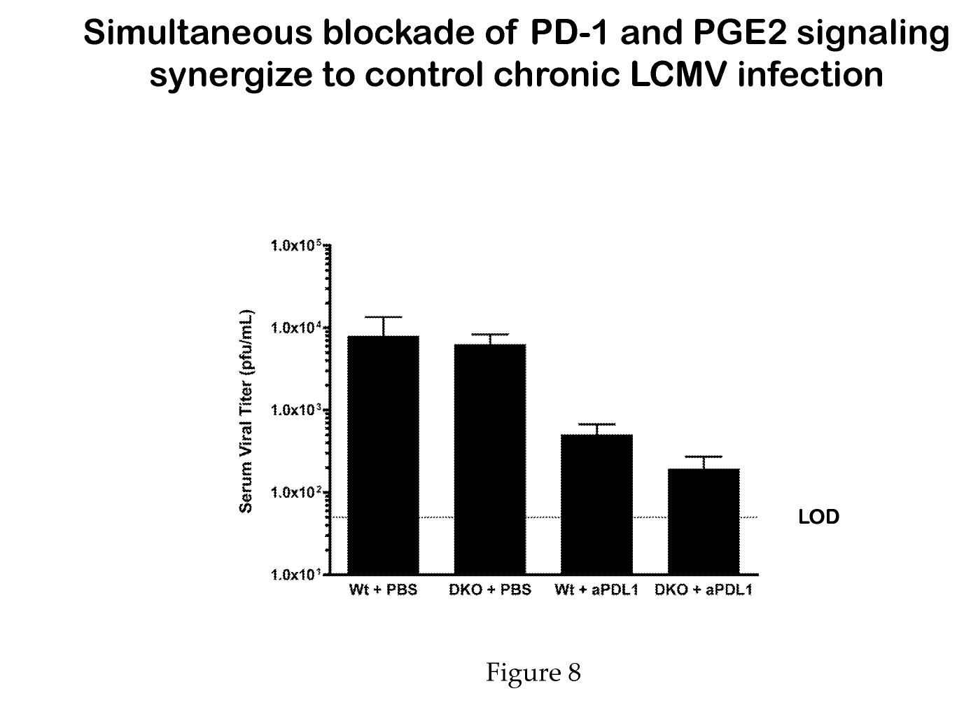

The concurrent inhibition of both PD-1 and PGE2 signaling synergistically increased the frequency and number of virus-specific CD8+ T cells at day 42 of LCMV infection. The inhibition of PD-L1 with anti-PD-L1 antibody increased the proportion of LCMV peptide-specific CD8+ T cells in anti-PD-L1 antibody treated EP2/EP4 DKO mice (15%) as compared with anti-PD-L1 antibody treated wt mice (1.6%) (FIG. 6). Moreover, the concurrent inhibition of both PD-1 and PGE2 signaling synergistically increased the production of INFγ, TNFα, and IL-2 by CD8+ T cells stimulated with LCMV peptides (i.e., NP396, GP33, or GP276) in anti-PD-L1 antibody treated EP2/EP4 DKO mice as compared with anti-PD-L1 antibody treated wt mice (FIG. 7). Additionally, the concurrent inhibition of both PD-1 and PGE2 signaling synergistically decreased serum viral titers in anti-PD-L1 antibody treated EP2/EP4 DKO mice as compared with anti-PD-L1 antibody treated wt mice (FIG. 8).

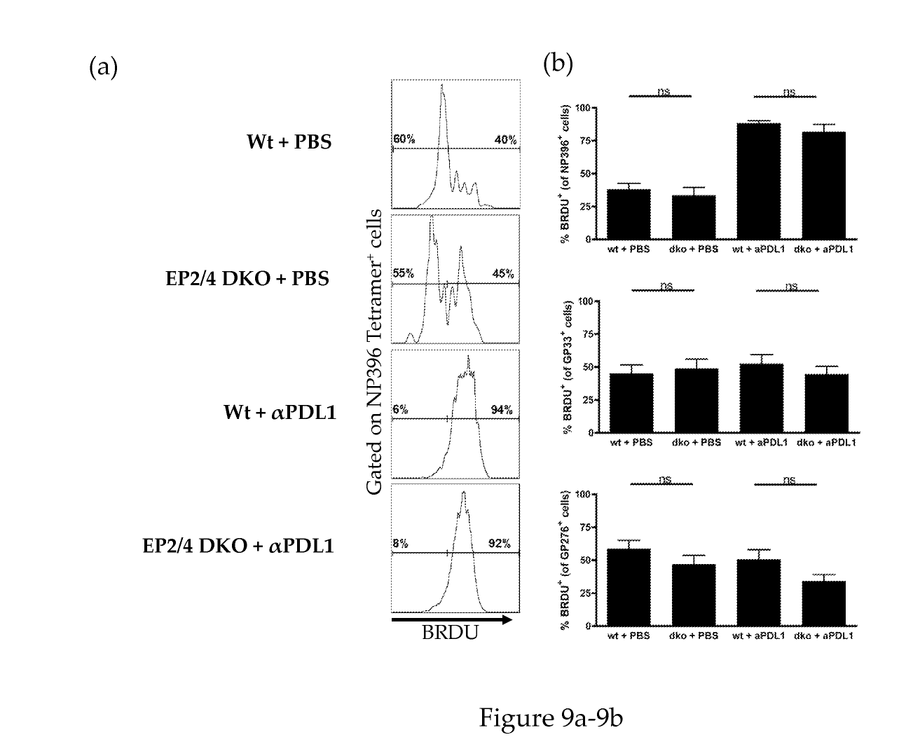

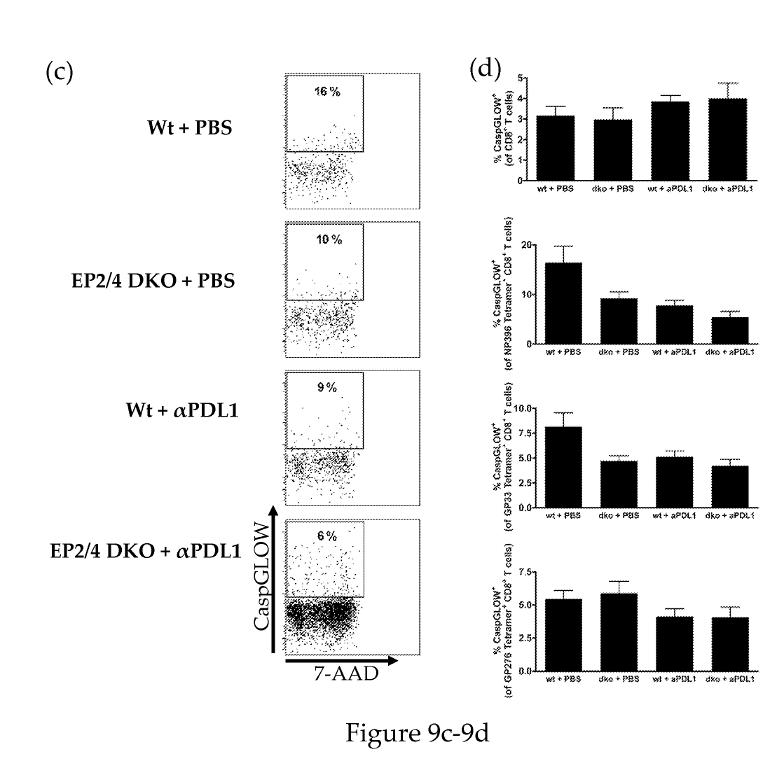

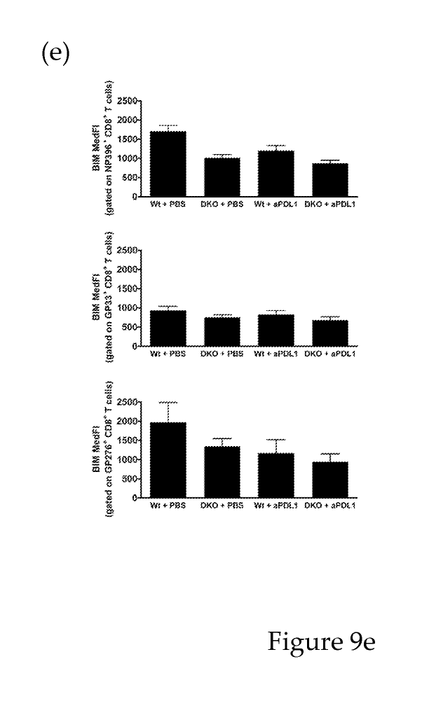

Furthermore, the concurrent inhibition of both PD-1 and PGE2 signaling augments CD8+ T cell proliferation and improves CD8+ T cell survival. EP2/4 DKO antigen-specific CD8+ splenic T cells do not appear to have a proliferative advantage, but may be less prone to apoptosis. The BRDU experiments included the addition of BRDU in the drinking water for the last week before sacrifice. Representative histograms for the NP396 tetramer-positive population (FIG. 9A) and compiled averages for all mice (FIG. 9B) are shown. For the results depicted in FIGS. 9c and 9d, cells were stained for active caspases with CaspGLOW to detect apoptosis. 7-AAD positive (dead) cells were excluded from the gate because these cells in this assay tended to non-specifically bind tetramer. Representative CaspGLOW FACS plots of for the NP396 tetramer-positive population (FIG. 9C) and compiled averages for all mice (FIG. 9D) are shown. Mean fluorescent intensity (MFI) of BIM staining for all mice were averaged for each of the indicated tetramers (FIG. 9E). These data are representative of two (three for BIM) experiments with at least three mice per group.

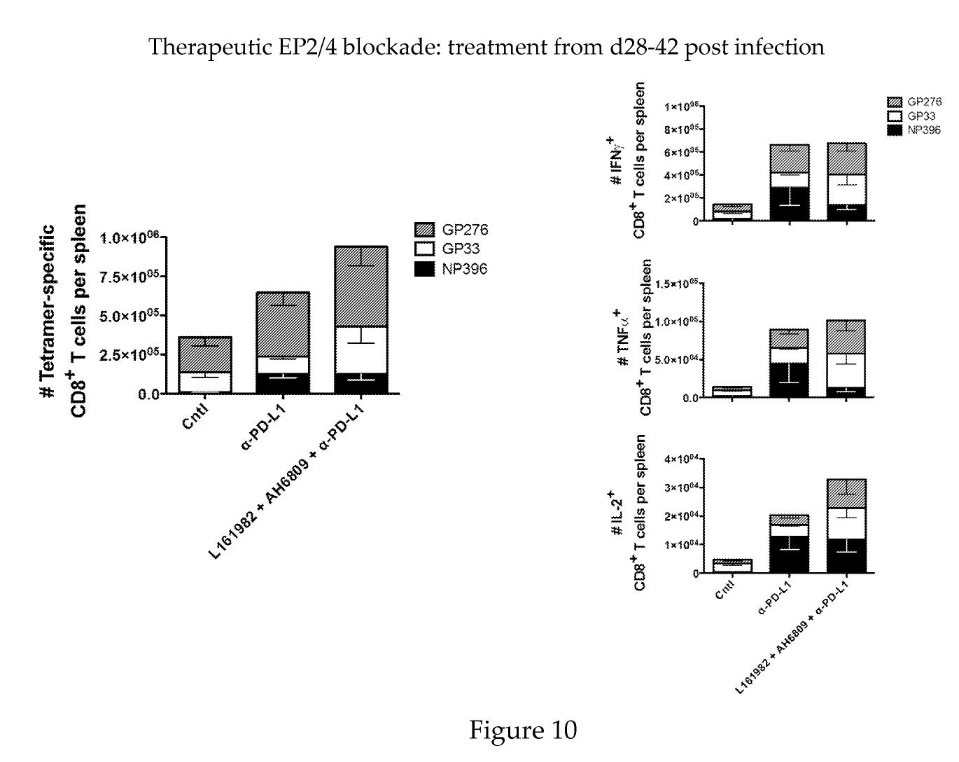

The concurrent inhibition of PD-1 signaling and EP2/EP4 signaling increased the number of virus-specific CD8+ T cells and increased INFγ, TNFα, and IL-2 production by CD8+ T cells when stimulated with the H-2D^{b}-binding LCMV peptides GP276, GP33 and NP396 (FIG. 10). Six week old female C57BL/6 mice were depleted of CD4+ T cells via administration of 200 μg GK1.5 antibody i.p. the day before and after LCMV CL13 infection (2×10⁶ PFU i.v.). One group of mice was treated with AH6809 (CAS 33458-93-4; an EP2 inhibitor; 200 ug per dose, oral gavage) and L161,982 (CAS 147776-06-5; an EP4 inhibitor; 200 ug per dose, i.p.) once daily beginning at day 28 post infection. This group and the “α-PD-L1” group received anti-PD-L1 antibody 200 μg i.p. every three days beginning at day 28 post infection. Mice were sacrificed at day 42 post infection. Antigen specific cells were counted using tetramer staining. Intracellular staining was performed after peptide stimulation in presence of brefeldin A. Data are compiled from 2 independent experiments of 3-4 mice per group.

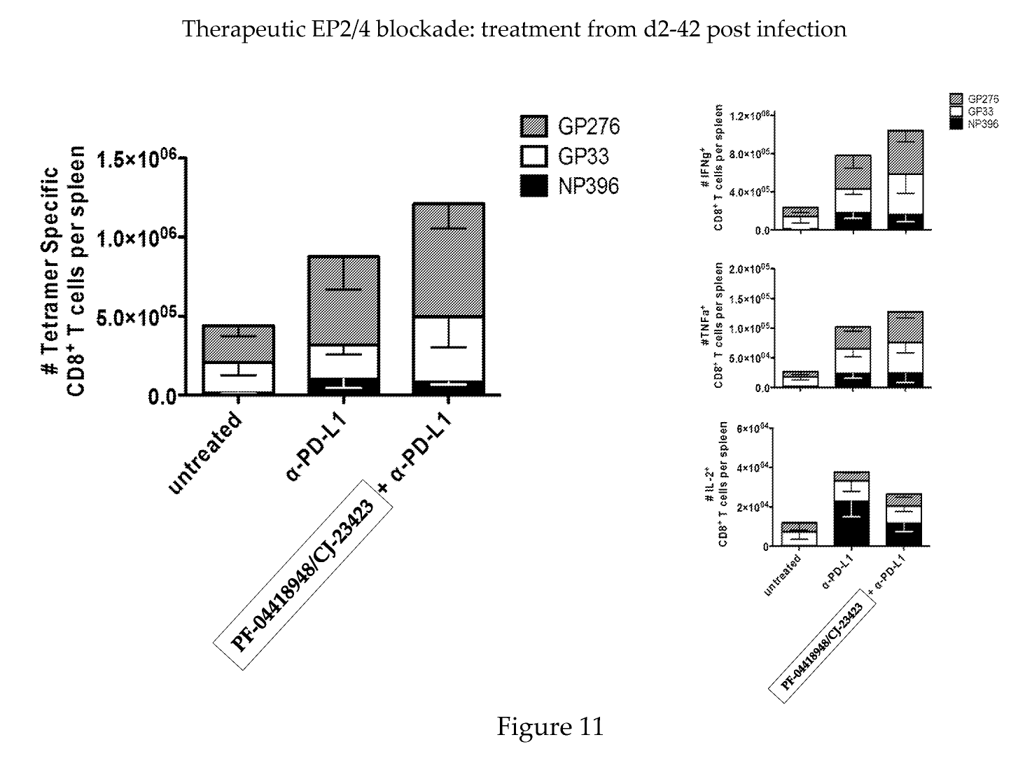

The concurrent inhibition of PD-1 signaling and EP2/EP4 signaling increased the number of virus-specific CD8+ T cells and increased INFγ, TNFα, and IL-2 production by CD8+ T cells when stimulated with the H-2D^{b}-binding LCMV peptides GP276, GP33 and NP396 (FIG. 11). Six week old female C57BL/6 mice were depleted of CD4+ T cells via administration of 200 μg GK1.5 antibody i.p. the day before and after LCMV CL13 infection (2×10⁶ PFU i.v.). One group of mice was treated with PF-04418948 (Forselles et al., 2011, Br. J. Pharmacol. 164:1847-56; an EP2 inhibitor; 200 ug per dose, oral gavage) and CJ-23423 (Hall et al., 2010, Burger's Medicinal Chemistry, Drug Discovery and Development: Agents acting on Prostanoid and Thromboxane Receptors, John Wiley & Sons; an EP4 inhibitor; 200 ug per dose, i.p.) once daily beginning at day 2 post infection. These mice were treated twice daily with these compounds at indicated doses from day 28-42 post infection. This group and the “a-PD-L1” group received anti-PD-L1 antibody 200 μg i.p. every three days beginning at day 28 post infection. Mice were sacrificed at day 42 post infection. Antigen specific cells were counted using tetramer staining. Intracellular staining was performed after peptide stimulation in presence of brefeldin A. Data are from a single experiment of four mice per group (except only two mice in untreated control group).

### Example 2

Chronic viral infections such as HIV, HCV, HBV are global health problems that collectively infect approximately 10% of the world's population and cause severe disease or death. The development of therapies to control or cure these infections without intolerable side effects is of obvious value. One such line of therapy is to augment the numbers and function of virus-specific CTLs via blockade of inhibitory signaling pathways. In the studies described herein, it is demonstrated that PGE2 is a factor that impairs CTL survival and effector functions during chronic LCMV infection. Blockade of PGE2 signaling either directly on the CTLs (via EP2/4 deletion) or systemically (via mPGES1 deletion) increased antigen-specific T cell numbers and their cytokine production, especially IL-2 production. The reduction PGE2 production in mice lacking mPGES1 also led to improved viral control when CD4 T cell help was absent, a situation typically associated with increased viremia and CTL exhaustion. Additionally, it is shown herein that simultaneous inhibition of both PGE2 and PD-1 signaling had cooperative and synergistic effects on controlling chronic viral infection, in part by restoring the immunodominant populations of virus-specific CD8 T cells (NP396- and GP34-specific CD8 T cells) that are deleted during LCMV-CL13 infection. These findings are significant because they identify a previously unrecognized role for PGE2 in suppressing CTLs during chronic viral infection, extending the list of negative regulatory factors (i.e., PD-1, LAG3, TIM3, TGFβ and IL-10) shown to be involved in these processes.

Additionally, the studies described herein are significant because they demonstrate cooperation between PGE2 and PD-1 and provide a rationale for combinational therapies targeting both pathways simultaneously. It is important that such treatments in chronic infections or in cancer are properly balanced to maximize T cell effector functions while minimizing the risk of immunopathology or autoimmunity. Notably, PD-1 blockade can be safely used in humans with minimal side effects, and this treatment is showing a high frequency of objective responses for advanced melanoma, renal cell carcinoma and non-small-cell lung cancer (Topalian et al., 2012, N Engl J Med 366:2443-2454). PGE2 blockade is likely to be a safe adjunctive therapy given that aspirin and NSAIDs such as ibuprofen have been used by hundreds of millions of people for decades with minimal side-effects, however the effects of these drugs on T cells during viral infections has received little clinical attention. In light of the studies described herein, exploring the use of NSAIDS or specific inhibitors of PGE2 signaling in combination with PD-1:PD-L1 blockade in clinical trials may lead to more robust effects.

The manner by which PD-1 and PGE2 suppress CTLs during chronic infection likely overlaps, but is also distinct to some degree. While anti-PD-L1 blockade greatly boosted CTL proliferation, PGE2 appeared to have a greater effect on CTL survival and Bim expression, a pro-apoptotic member of the Bcl-2 family.

Another manner by which PGE2 signaling may suppress CTL survival during chronic viral infection is through the repression of IL-2 production. Reduced IL-2 production is a notable feature of CTL exhaustion that precedes the loss of TNFα and IFNγ production and is necessary for maintenance of virus-specific CD8 T cells during chronic LCMV infection (Wherry et al., 2003, J Virol 77:4911-4927). It is shown herein that abolishing PGE2 signaling increased IL-2 expression in CD8 T cells during LCMVCL13 infection, which was particularly evident in the population of NP396-specific CTLs. Although the production of IL-2 by CD4 T cells helps CTLs during chronic viral infection, it is also possible that CD8 T cell autonomous production of IL-2 is also important.

Here, several approaches are employed to show that PGE2 signaling directly suppresses exhausted antigen-specific CD8 T cell function and promotes their apoptosis. Furthermore, a link between PGE2 and PD-1 signaling is described. In the setting of LCMV-CL13 infection, mice deficient in PGE2 signaling were far more responsive to anti-PD-L1 blockade than their Wt counterparts, indicating a synergistic relationship between PGE2 and PD-1 signaling. This synergy appeared to result from increased TCR signal and decreased Bim levels. Thus, comodulation of PGE2 and PD-1 signaling represents a potent therapeutic avenue in the treatment of chronic disease.

The materials and methods used in this Experimental Example are now described.

### Mice, Infections, Treatments, and Plaque Assays

Six week old female C57BL/6 mice were obtained from NCI (Frederick, Md.). EP2 KO mice (Kennedy et al., 1999, Nat. Med. 5:217-220) on the C57BL/6 line were a gift from Dr. Chuanming Hao. Genotyping for the wild-type (wt) allele used the primers 5′-CCGGGGTTCTGGGGAATC-3′ (SEQ ID NO: 1) and 5′-GTGCATGCGAATGAGGTTGAG-3′ (SEQ ID NO: 2). Genotyping for the mutant allele used the primers 5′-TTGCCAAGTTCTAATTCCATCAGA-3′ (SEQ ID NO: 3) and 5′-GTGCATGCGAATGAGGTTGAG-3′ (SEQ ID NO: 4). EP4-floxed mice (Schneider et al., Genesis 40:7-14) on the C57BL/6 line were also a gift from Dr. Chuanming Hao. Genotyping the EP4-floxed mice used the primers 5′GTTAGATGGGGGGAGGGGACAACT-3′ (SEQ ID NO: 5) and 5′TCTGTGAAGCGAGTCCTTAGGCT-3′ (SEQ ID NO: 6). The floxed gene produced a 334 bp band, while the wt allele produced a 243 bp band. mPGES1−/− mice (Trebino et al., Proc Natl Acad Sci USA 100:9044-9049) on the C57BL/6 line were a gift from Dr. Daniel Rosenberg. Genotyping for the mutant allele used the primers 5′-GGAAAACCTCCCGGACTTGGTTTTCAG-3′ (SEQ ID NO: 7) and 5′-ATCGCCTTCTATCGCCTTCTTGACGAG-3′ (SEQ ID NO: 8). While genotyping for the wt allele used the primers 5′-GGAAAACCTCCCGGACTTGGTTTTCAG-3′(SEQ ID NO: 9) and 5′-CAGTATTACAGGAGTGACCCAGATGTG-3′ (SEQ ID NO: 10). EP4-floxed mice were crossed to Granzyme B-cre mice (Jacob and Baltimore, Nature 399:593-597) and then to the EP2−/− mice to create EP2/4 DKO mice.

EP2/4 DKO mice were crossed to Thy1.1+ P14 TCR transgenic (tg) mice (Kaech and Ahmed, Nat. Immunol. 2:415-422). 5,000 CD8+ Thy1.1+ P14 TCR tg cells were transferred i.v. into wt C57BL/6 mice to create “P14 chimeric mice” (Joshi et al., 2007, Immunity 27:281-295). C57BL/6 mice used as controls for all of the knockouts were derived from wt littermates to the knockout strains. Importantly, these knockout mice were housed in the same room of the mouse facility as the knockout mice. Mice were infected with 2×10⁵ PFU of LCMV-Armstrong (i.p.) or 2×10⁶ PFU of LCMV-CL13 (i.v.) as described by Joshi (Joshi et al., 2007, Immunity 27:281-295). For indicated experiments, mice were depleted of CD4+ T cells by administration of anti-CD4 antibody (clone GK1.5 at dose of 200 μg intraperitoneally) the day before and after infection. All animal experiments were done with approved Institutional Animal Care and Use Committee protocols.

Two sets of EP2 and EP4 inhibitor compounds were used (see Figures for each set for the particular experiment). The first set was AH6809 (EP2 blocking) (CAS 33458-93-4, Cayman Chemical, Ann Arbor, Mich.) and L161,982 (EP4 blocking) (CAS 147776-06-5, Cayman Chemical, Ann Arbor, Mich.). The second set was with PF-04418948 (EP2 blocking) (Pfizer, Groton, Conn.; see, for example, of Forselles, 2011, Br J Pharmacol. 164:1847-56) and CJ-23423 (EP4 blocking) (Pfizer, Groton, Conn.). For both sets of EP2 inhibitor compounds, the inhibitor compound was administered by oral gavage 200 μg in 100 μL corn oil). For both sets of EP4 inhibitor compounds, the inhibitor compound was administered i.p. (200 μg in 100 μL PBS). Control animals were given gavages of 100 μL corn oil and i.p. injections of 100 μL PBS. In cases where anti-PD-L1 (Bio X Cell, West Lebanon, N.H.) was administered, mice were i.p. injected with 200 μg of antibody in 200 μL of PBS.

### Cell Isolation, Surface, and Intracellular Staining

BrdU (Sigma, St. Louis, Mo.) was administered at 1 mg/mL in the drinking water. Detection of BrdU was performed following the instructions on the staining kit (BD, San Jose, Calif.). CaspGLOW staining was performed according to provided instructions (eBioscience, San Diego, Calif.). Tetramer staining and T cell stimulation with LCMV peptide was performed as previously described (Fuller et al., J. Immunol. 172:4204-4214). To calculate relative fluorescence, PD-1 MFIs were averaged for DbGP33 tetramer-positive cells from the vehicle-treated Wt mice within each replicate experiment. This average MFI was divided by 10,000 to generate a scaling factor by which the absolute PD-1 MFIs for each sample within a given replicate could be multiplied to generate a normalized relative PD-1 MFI. Intracellular staining for Bim (Cell Signaling Technology, Boston, Mass.) was performed using FoxP3 Fix/Perm Buffer (BioLegend, San Diego, Calif.) and Perm/Wash Buffer (BD, San Jose, Calif.). PGE₂ (Cayman Chemical, Ann Arbor, Mich.) was administered at 40 μM at the time of peptide administration for the indicated in vitro stimulations. Flow cytometry was performed on a LSRII machine (BD, San Jose, Calif.). Flow cytometry analysis was performed using FlowJo software (Tree Star, Ashland, Oreg.). Sorting was performed on a FACS Aria (BD, San Jose, Calif.).

Gene Expression by qRT-PCR and PGE2 Measurements

For qRT-PCR, RNA was isolated from 500,000 sorted cells following the instructions provided with the Qiashredder and RNeasy kits (Qiagen, Valencia, Calif.). cDNA was then synthesized using SSRTII (Invitrogen, Carlsbad, Calif.). Real time was performed on a Stratagene Mx3000P, as previously described (Joshi et al., 2007, Immunity 27:281-295). Relative fold changes were calculated using L9 expression as a normalization constant.

PGE2 measurement was performed by ELISA (Cayman Chemical, Ann Arbor, Mich.). For PGE2 measurement, media was harvested from cultures 24 hours after they were set up. Media was spun to precipitate out cellular debris, and the resulting supernatant was stored at −80° C. until its use in the PGE2 ELISA.

Viral plaque assays were performed following von Herrath's protocol (von Herrath and Whitton, 2003, Animal Models Using Lymphocytic Choriomeningitis Virus. Current Protocols in Immunology, Unit 19.10). Briefly, Vero cells were plated in 6 well plates. When the Vero monolayer was ˜60% confluent, a dilution of either serum or tissue homogenate sample was added to each well. The virus in each sample was then given one hour to infect the Vero monolayer. Thereafter, an agarose gel containing serum, 199 media, and Seakem agarose was placed over the monolayer. These 6 well plates were then incubated for four days in a CO₂ incubator. After this period, formaldehyde was added to the wells and the plates were allowed to sit for 2 hours. Then, the agarose gel was removed and a 0.1% solution of crystal violet was added to each well. After 2 hours, the plates were washed in water. Plaques were then counted and the viral load was back-calculated using the volume of sample added and its dilution.

### Statistical Analysis

Statistical analysis was performed using unpaired two-tailed t test when comparing two groups. Analysis of three or more groups was performed using ANOVA, followed by unpaired two-tailed t test post-test of pairings identified as significant by ANOVA. Analysis of anti-PD-L1 treatment versus genotype data used two-way ANOVA. For two-way ANOVA analyses involving cell numbers, viral titers, and MFI, a log transformation of each datum was performed to normalize each data set prior to running the ANOVA.

The results of this Experimental Example are now described.

### The PGE2 Receptors, EP2 and EP4, are Upregulated on Virus-Specific CD8 T Cells During Chronic LCMV Infection

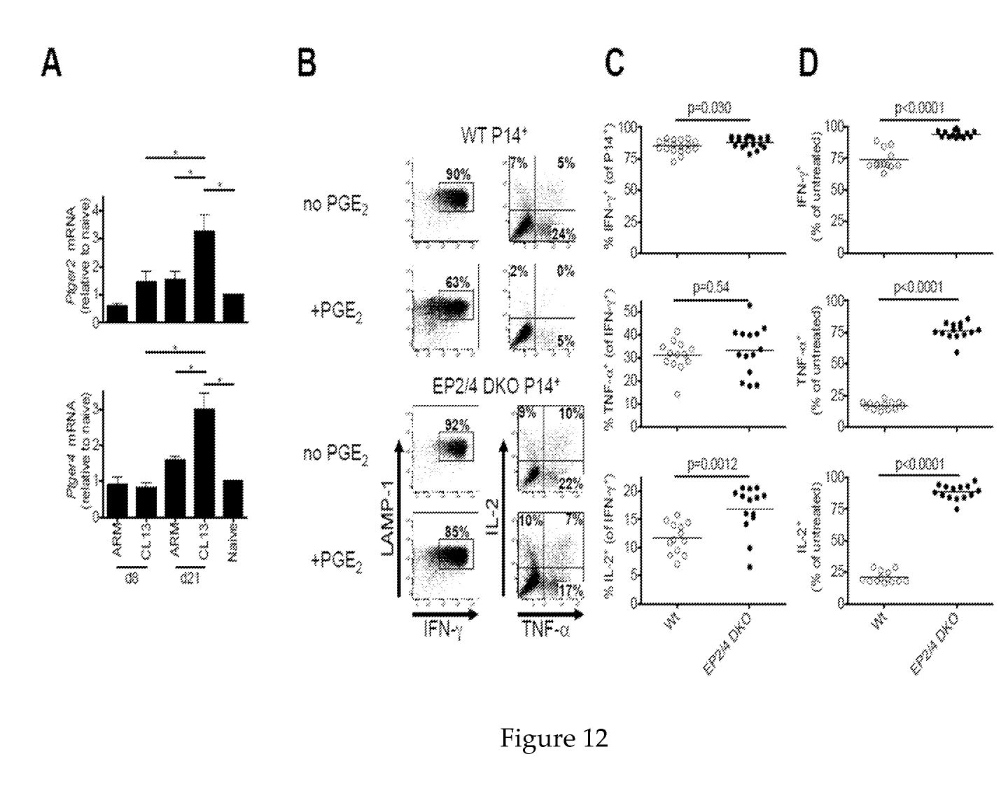

Whole genome expression profiling showed that virus-specific CD8 T cells upregulated the PGE2 receptors Ptger2 (EP2) and Ptger4 (EP4) during chronic LCMV-CL13 infection. To validate and extend this finding, splenic LCMV-specific CD8 T cells were sorted on days 8 and 21 post infection (p.i.) with acute LCMV-Arm or chronic LCMV-CL13, and the amounts of Ptger2 and Ptger4 mRNA were measured using qRT-PCR. First, this showed that Ptger2 and Ptger4 expression increased ˜3-fold over the duration of LCMV-CL13 infection (by day 21 p.i.) compared to naïve CD8 T cells (FIG. 12A). Second, consistent with the gene expression profiling data, Ptger2 and Ptger4 mRNA were higher in CD8 T cells isolated at day 21 p.i. from chronic LCMV-CL13 infection relative to those from acute LCMV-Arm infection, indicating that these genes are more strongly upregulated in the setting of chronic infection.

### PGE2 Signals Through its Receptors EP2 and EP4 on CD8 T Cells to Directly Suppress Effector Functions in LCMV-Specific CD8 T Cells

PGE2 has been shown to suppress proliferation of activated CD8 T cells in vitro (Obermajer et al., 2011) and has been suggested to suppress their cytokine production as well (Pettersen et al., 2011). Therefore, the effects of PGE2 on virus-specific CD8 T cell effector functions was assessed. Virus-specific CD8 T cells were generated that lack both EP2 and EP4 by intercrossing Ptger2−/− mice with those possessing floxed alleles of EP4 (Ptger4fl/fl) and a transgene that expresses cre recombinase under the Granzyme B (GzmB) promoter (GzmB-cre) to create Ptger2−/−;Ptger4fl/fl;GzB-cre+ mice (herein referred to as “EP2/4 DKO”). In these animals, all cells lack EP2, but only cells that express granzyme B, such as virus-specific CD8 T cells, lack both EP2 and EP4. The EP2/4 DKO mice were then crossed to the P14+TCR transgenic mouse strain (which expresses a TCR that recognizes the LCMV GP33-41 peptide) to create P14+ EP2/4 DKO mice. Ptger2+/+;Ptger4+/+;GzB-cre-mice were used as littermate controls and are referred to as “Wt” herein.

To examine the effects of PGE2 on effector CD8 T cell functions, Wt or EP2/4 DKO P14+ CD8 T cells were transferred into C57BL/6 mice that were subsequently infected with LCMV-CL13. Eight days later, the mice were sacrificed and the P14+ CD8 T cells were stimulated with GP33 peptide and the production of IFN-γ, TNF-α and IL-2 was measured by intracellular cytokine staining (ICCS).

Similar percentages of IFN-γ and TNF-α producing cells were observed between the Wt and the EP2/4 DKO P14+ CD8 T cells, and a greater proportion of EP2/4 DKO cells produced IL-2 (FIGS. 12B and 12C). The effects of PGE2 on cytokine production were further assessed by adding PGE2 to the ICCS. The treatment of PGE2 on Wt P14+ CD8 T cells suppressed production of all three cytokines with the greatest effects observed on TNF-α and IL-2 production (˜75-80% inhibition) (FIGS. 12B and 12D). In contrast, PGE2 had very little effect on cytokine production by the EP2/4 DKO cells, indicating that deletion of EP2 and EP4 abolishes PGE2 signaling in virus-specific CD8 T cells (FIGS. 12B and 12D). Thus, these results demonstrate that PGE2 directly suppressed virus-specific CD8 T cell cytokine production in an EP2- and EP4-dependent manner.

### PGE2 Suppresses Virus-Specific CD8 T Cell Numbers and Function During Chronic LCMV Infection

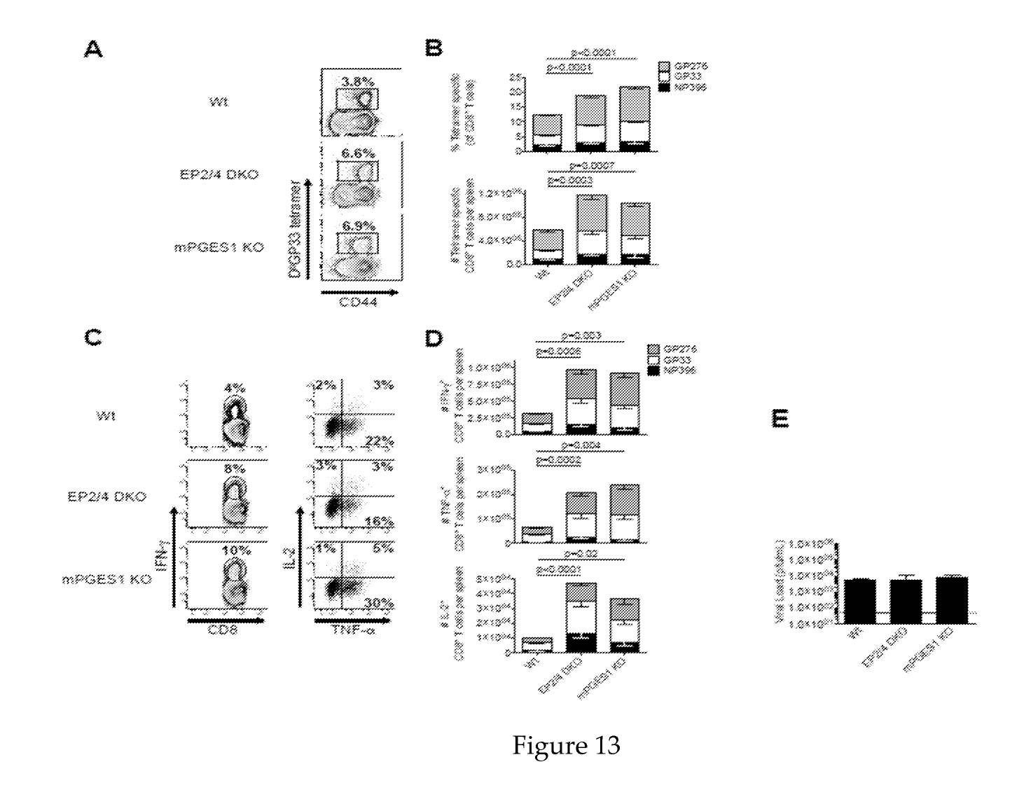

To extend these findings to chronic viral infection, whether PGE2 suppressed CTL responses during chronic LCMV-CL13 infection was next examined. In addition to the EP2/4 KO mice, mice deficient in mPGES 1 (herein referred to as “mPGES1 KO”) were employed to study the effects of reducing PGE2 systemically during LCMV-CL13 infection. Wt, EP2/4 DKO, and mPGES1 KO mice were infected with LCMV-CL13 and then sacrificed at day 21 p.i. for analysis of the virus-specific CD8 T cells. MHC class I tetramers were used to measure the numbers of NP396-, GP276- and GP33-specific CD8 T cells. A 1.8-2 fold increase was observed in the frequency and number of the virus-specific CD8 T cells in EP2/4 DKO and mPGES1 KO mice compared with the Wt mice at day 21 p.i. (FIGS. 13A and 13B). These findings indicate that PGE2 directly suppressed the expansion of virus-specific CD8 T cells during chronic viral infection.

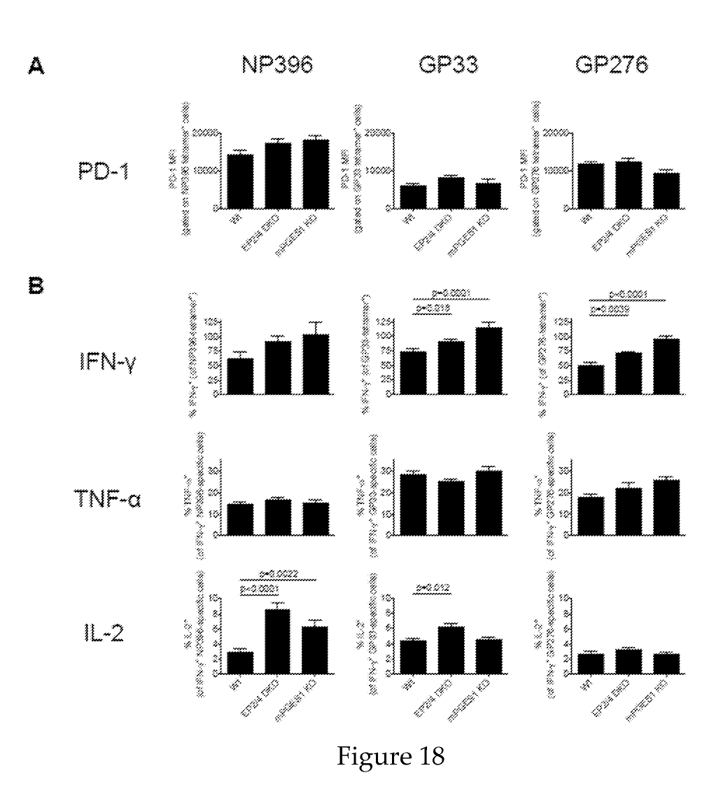

The ability of the three different virus-specific CD8 T cell populations to produce IFN-γ, TNF-α and IL-2 in response to peptide stimulation was then assessed. An increase in the percentage and number of cytokine producing cells in the EP2/4 KO and mPGES1 KO mice relative to the Wt mice (FIGS. 13C, 13D and 18B) was observed. The number of cells producing IFN-γ and TNF-α was increased ˜2-3 fold and the number of IL-2 producing cells was augmented even more (˜4-5 fold) in the animals with reduced PGE2 signaling. Together, these data demonstrate that PGE2 signaling suppressed CTL cytokine production and contributed to their functional exhaustion.

### PD-1 is Expression is Increased in EP2/4 DKO CD8 T Cells

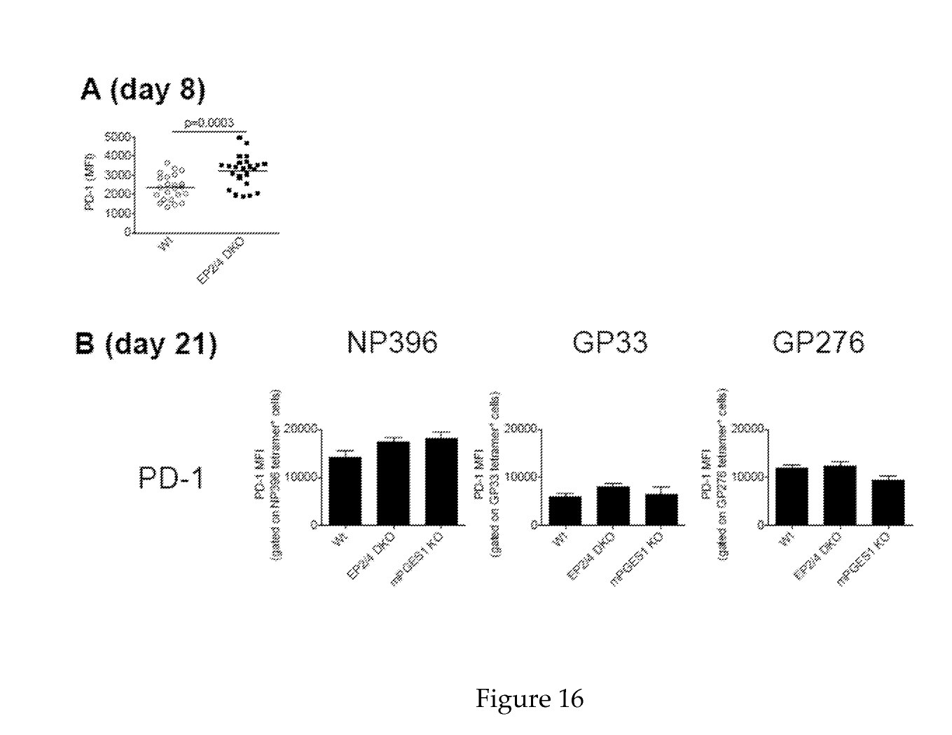

During LCMV-C113 infection, several inhibitory receptors are upregulated on the virus-specific CD8 T cells, including PD-1, LAG3, CD160 and 2B4. Although not wishing to be bound by any particular theory, this is consistent with the explanation that the PD-1 inhibitory pathway may be compensating, at least in part, for the lack of PGE2 signaling. To investigate this possibility, surface PD-1 expression was compared on EP2/4 DKO cells to WT cells. Interestingly, it was found that the expression of PD-1 was increased in the EP2/4 KO virus-specific CD8 T cells relative to their Wt counterparts at both day 8 and 21 p.i., although all strains of mice exhibited similar viral titers (FIG. 16). These data suggest that in spite of increased CTL numbers and function, elevated PD-1 expression may compensate for the loss of PGE2 signaling and prevent elimination of viral infected cells.

### Combined Blockade of PGE2 and PD-1 Signaling Greatly Boosts Antiviral T Cell Responses

Studies were conducted to examine whether the blockade of two or more inhibitory pathways might have synergistic effects toward improving CTL responses during LCMV-CL13 infection. As noted earlier, because PD-1 was increased on EP2/4 DKO CD8 T cells, whether the simultaneous blockade of PD-1 and PGE2 signaling could augment CTL responses and viral control during LCMV-CL13 infection more than blockade of either one alone was examined Wt, EP2/4 DKO or mPGES1 KO mice were depleted of CD4 T cells using GK1.5 (anti-CD4) mAb treatment, infected with LCMV-CL13 and then treated with anti-PD-L1 mAb or vehicle control from days 28-42 p.i. The loss of CD4 T cell help causes persistent high titer viremia, rapid depletion of NP396-specific CD8 T cells and greater functional exhaustion (Zajac et al., 1998, J Exp Med 188:2205-2213).

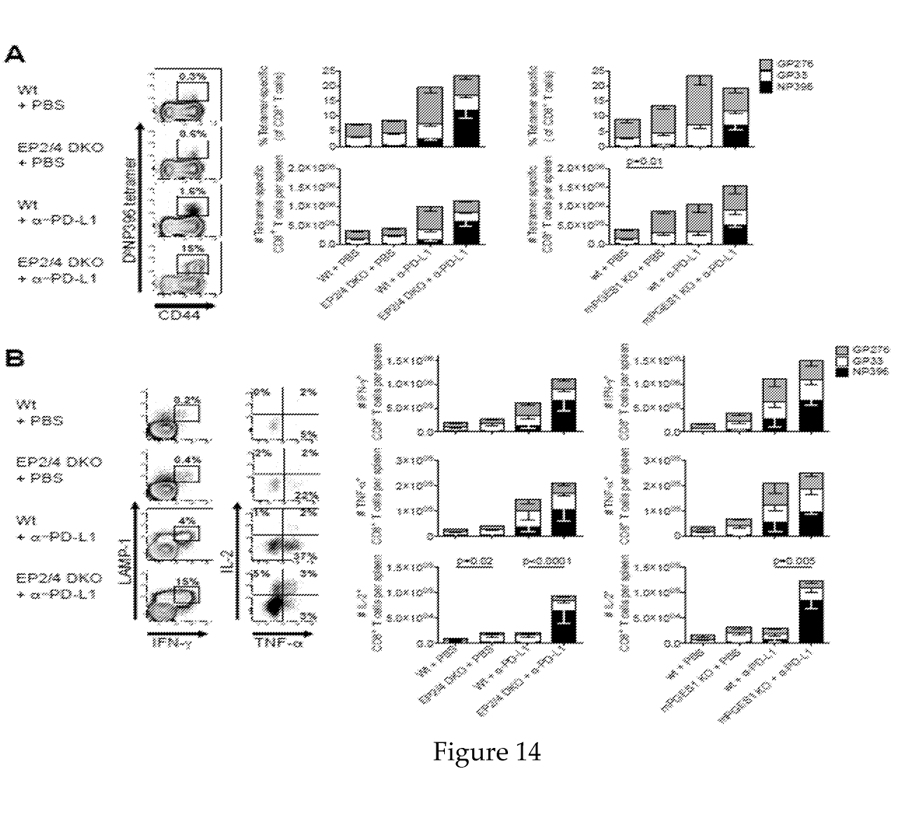

Similar to the CD4-replete setting described above, the systemic reduction of PGE2 in the mPGES1 KO mice led to an increase (˜2-fold) in the number of functional virus-specific CD8 T cells as compared to Wt mice in the absence of CD4 T cell help in LCMV-CL13 infected mice (FIG. 14A). However, there was no observable effect on virus-specific CD8 T cell numbers in the EP2/4 DKO mice in the ‘helpless’ system (FIG. 14A). Wt mice responded to anti-PD-L1 treatment and increased numbers of LCMV-specific CD8 T cells were observed relative to the vehicle-treated Wt controls (FIG. 14A). Of the virus-specific CD8 T cell populations analyzed (i.e., NP396, GP33, and GP276), anti-PD-L1 blockade in Wt mice caused expansion of the GP276- and GP33-specific CD8 T cells to a considerably greater extent than the NP396-specific CD8 T cells (FIG. 14A). PD-L1 blockade also increased the number of IFN-γ and TNF-α producing CD8 T cells, but little effect on IL-2 production was noted (FIG. 14B). Thus, suppressing PD-L1 increased the number of functional virus-specific CD8 T cells, but it did not rescue deletion of the NP396-specific CTLs or boost IL-2 producing cells to a considerable extent.

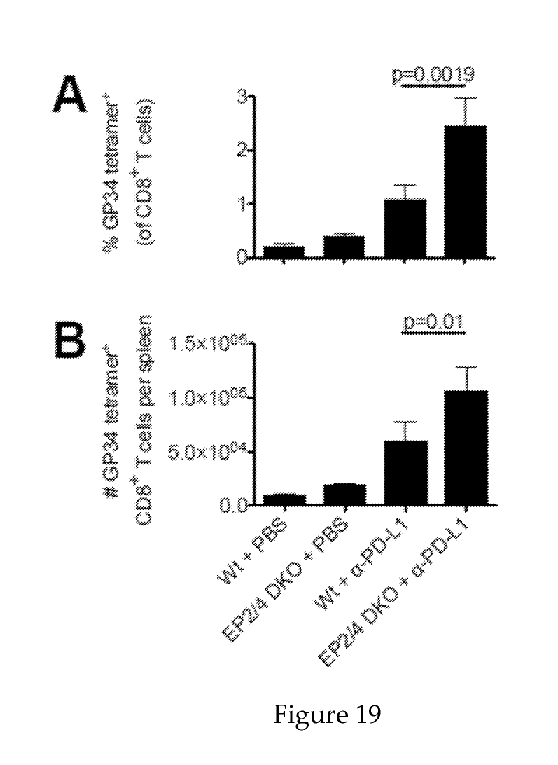

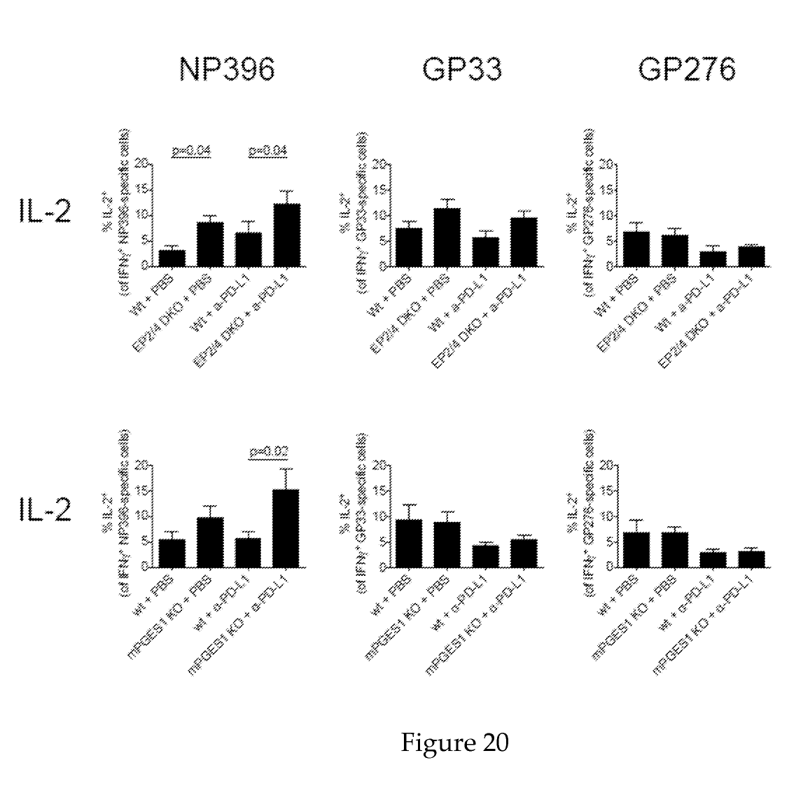

Interestingly, the effects of anti-PD-L1 blockade were enhanced significantly in mice deficient in PGE2 signaling. In particular, PD-L1 blockade in the EP2/4 DKO or mPGES1 KO mice boosted the NP396-specific CD8 T cell population by 5-fold relative to the Wt mice (FIG. 14A). Blockade of PD-L1 in the EP2/4 DKO mice also greatly boosted the numbers of GP34-specific CD8 T cells (FIG. 19), which is another population of virus-specific CD8 T cells that is normally deleted during LCMV-CL13 infection (Wherry et al., 2003, J Virol 77:4911-4927). These data demonstrate that blockade of PGE2 signaling complemented the effects of anti-PD-L1 therapy and considerably helped to restore the populations of virus-specific CD8 T cells that are typically deleted during chronic LCMV-CL13 infection.

Next, the effects of a combination blockade of PD-L1 and PGE2 signaling on cytokine production by antiviral CD8 T cells was assessed by ICCS. Blockade of PD-L1 alone potently boosted the frequency and number of IFN-γ- and TNF-α-producing cells (FIG. 14B). However, its effects were enhanced when combined with concomitant reduction in PGE2 signaling, particularly in the NP396-specific CD8 T cell population. Notably, the biggest effect was on IL-2 production; the number of IL-2-producing CD8 T cells was four-fold higher in the EP2/4 DKO and mPGES1 KO mice treated with anti-PD-L1 compared to the Wt mice (FIG. 14B). Interestingly, the NP396-specific cells constituted the majority of the IL-2 producers in the anti-PD-L1 treated EP2/4 DKO and mPGES1 KO mice demonstrating that reduction of PGE2 signaling not only helped this CD8 T cell population persist, but also helped to sustain their effector functions (FIG. 14B).

### Combination Blockade of PD-L1 and PGE2 Signaling Enhances Control of Chronic Viral Infection

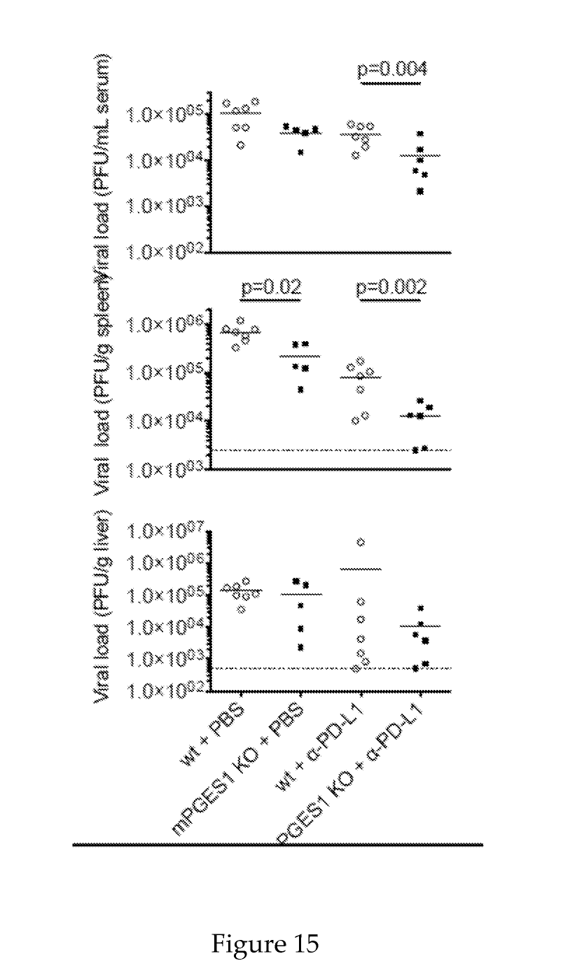

To determine if the combined effects of reduced PD-1:PD-L1 and PGE2 signaling enhanced viral clearance in the absence of CD4 T cell help, viral titers were measured in the serum, spleen and liver at day 42 p.i. in the different groups of mice. As expected, blockade of PD-L1 alone enhanced viral clearance. Significant reductions in the viral titers in the serum and spleen were also observed in mPGES1 KO mice compared to their Wt counterparts. There was also a trend towards lower viral titers in the livers of mPGES1 KO mice (FIG. 15). Importantly, the effects of anti-PD-L1 blockade on viral clearance were further enhanced in the mPGES1 KO mice, consistent with the increased numbers of functional antiviral CD8 T cells observed above FIG. 15). Compared to the Wt (vehicle treated) controls, the combination of reducing PGE2 and PD-1 signaling simultaneously, decreased viral titers by as much as 10-100 fold depending on the tissue examined

### PGE2 Promotes Bim Expression in Exhausted Antigen-Specific CD8 T Cells

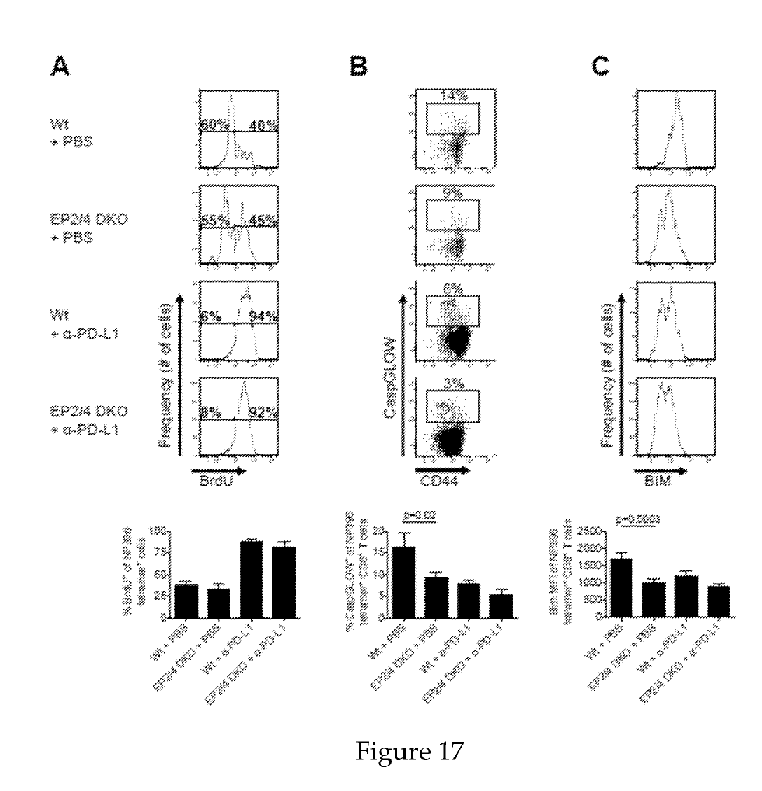

Whether the increased number of NP396- and GP34-specific CD8 T cells in the anti-PD-L1 treated EP2/4 DKO mice was due to enhanced CD8 T cell division, survival, or both, was examined next. First, T cell division was measured by BrdU staining. CD4-depleted Wt or EP2/4 DKO mice with or without anti-PD-L1 treatment received BrdU in their drinking water during the second week of anti-PD-L1 treatment. The NP396-specific CD8 T cells divided at similar rates in the vehicle-treated Wt and EP2/4 DKO mice (˜40-50% of the cells incorporated BrdU), indicating reduction of PGE2 signaling alone had little effect on CD8 T cell division (FIG. 17A). The rates of cell division increased greatly when PD-1:PD-L1 signaling was blocked (>90% of the cells incorporated BrdU), however, there was no additive effect of blocking PGE2 and PD-1 signaling simultaneously (FIG. 17A). These data suggest that PGE2 signaling was not suppressing proliferation of the virus-specific CD8 T cells during chronic viral infection.

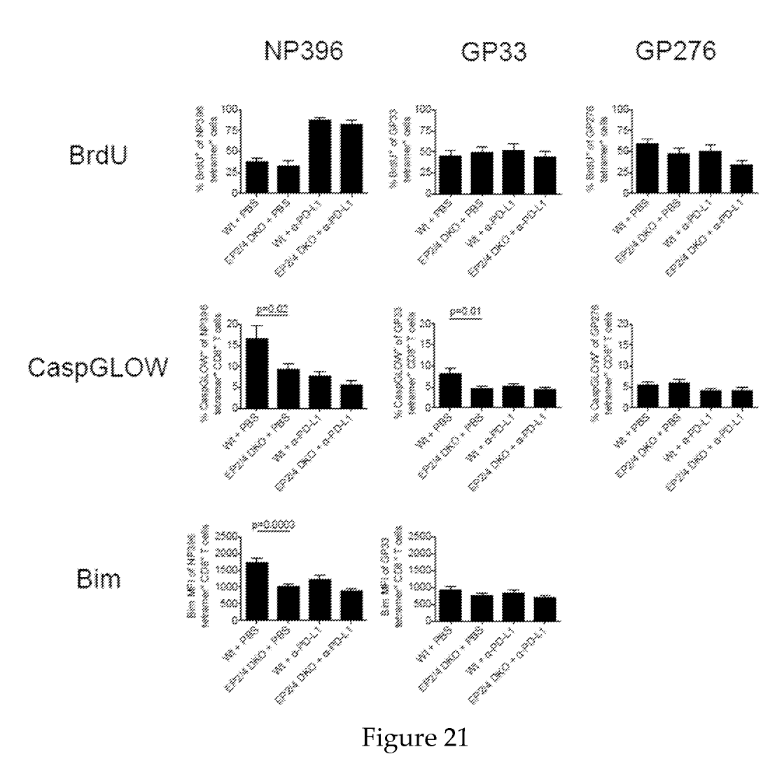

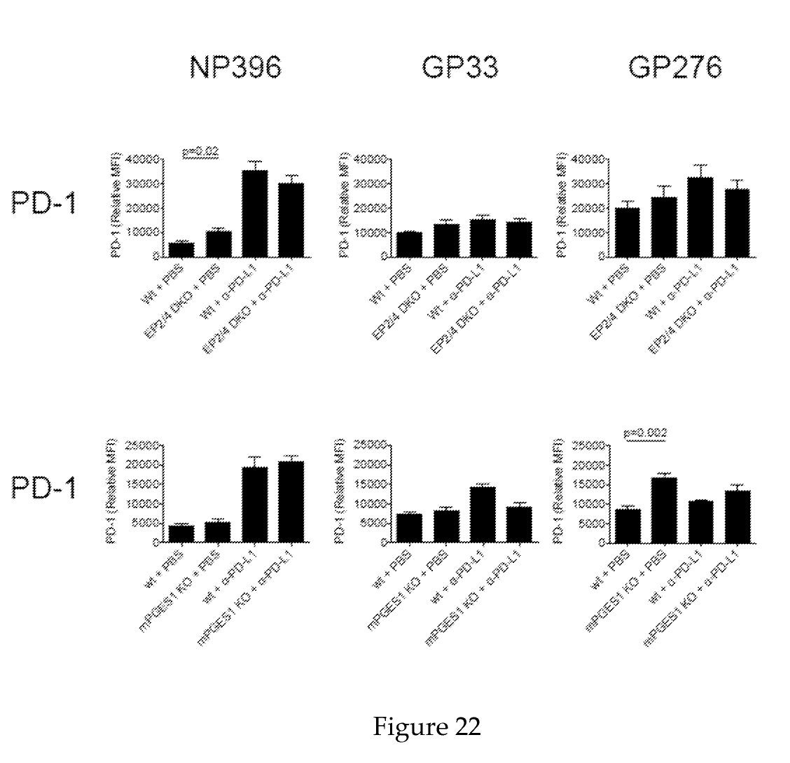

Next, the frequency of CD8 T cells undergoing apoptosis in the CD4-depleted Wt or EP2/4 DKO mice with or without anti-PD-L1 blockade was assessed by staining the cells with antibodies to Bim, as well as CaspGLOW reagents that bind caspase-3 in apoptotic cells (Callus and Vaux, 2007, Cell Death Differ. 14:73-8). Based on these parameters, NP396-specific CD8 T cells in EP2/4 DKO mice exhibited significantly lower rates of apoptosis than their Wt counterparts (FIGS. 17B and 17C). The frequency of CaspGLOW+NP396-specific CD8 T cells and the amount of Bim they contained in the EP2/4 DKO mice was approximately half that of the Wt controls (FIGS. 17B and 17C). A similar reduction in apoptotic cells was observed in mice treated with anti-PD-L1 (FIGS. 17B and 17C). There was decrease in the percentage of apoptotic cells in the NP396-specific CD8 T cells in EP2/4 DKO versus Wt mice treated with anti-PD-L1 (FIGS. 17B and 17C). The effect on apoptosis of GP33-specific CD8 T cells by blocking either PGE2 or PD-L1 signaling was demonstrable, but less profound, and little to no effect was observed in GP276-specific CD8 T cells (FIG. 21). These results indicate that blocking either PGE2 or PD-1 signaling alone reduced Bim expression and enhanced survival of virus specific CD8 T cells during chronic LCMV infection, but there was no synergistic effect of reducing both PGE2 and PD-L1 signaling simultaneously. Together, these results indicate that PGE2 plays a more dominant role in suppressing antiviral CD8 T cell survival than cell proliferation. Therefore, although not wishing to be bound by any particular theory, the observed synergy between PGE2 and PD-1 blockade is consistent with the explanation that it results from simultaneous increase in T cell division resulting from PD-1 blockade and decrease in T cell death secondary to loss of PGE2 signal.

### Combination Blockade of PD-L1 and PGE2 Signaling Enhances Antiviral CTL Response

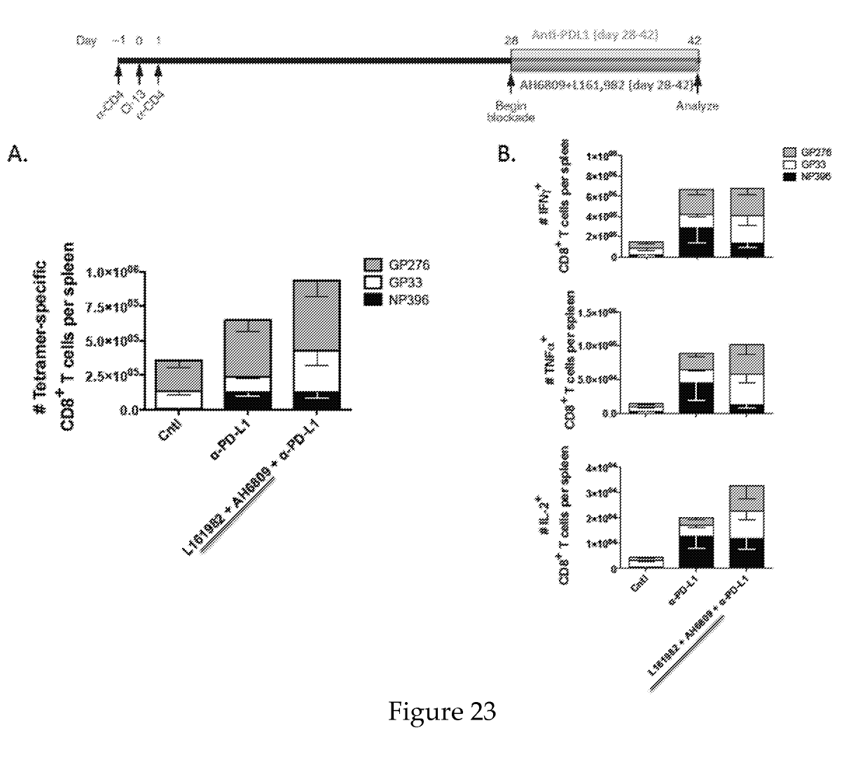

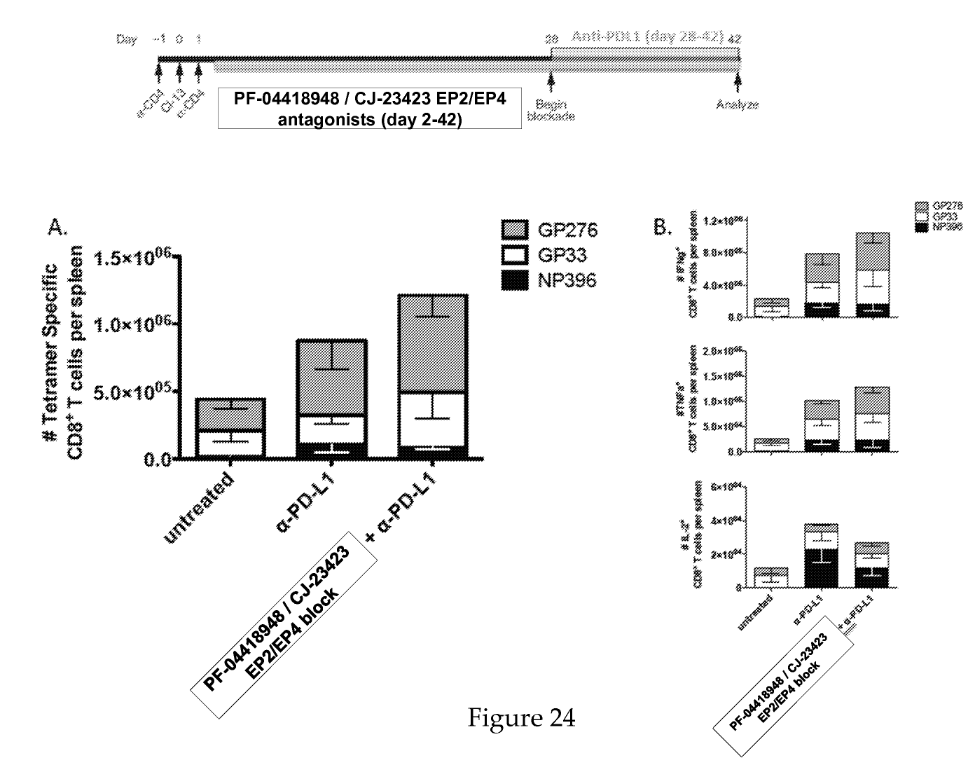

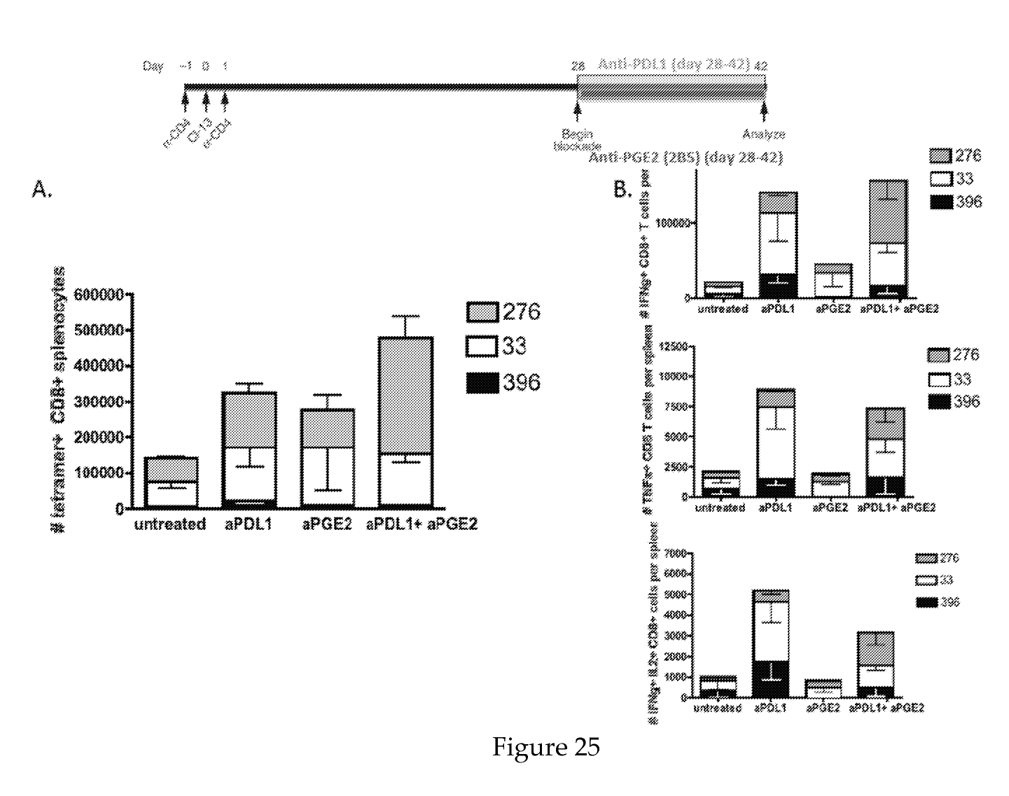

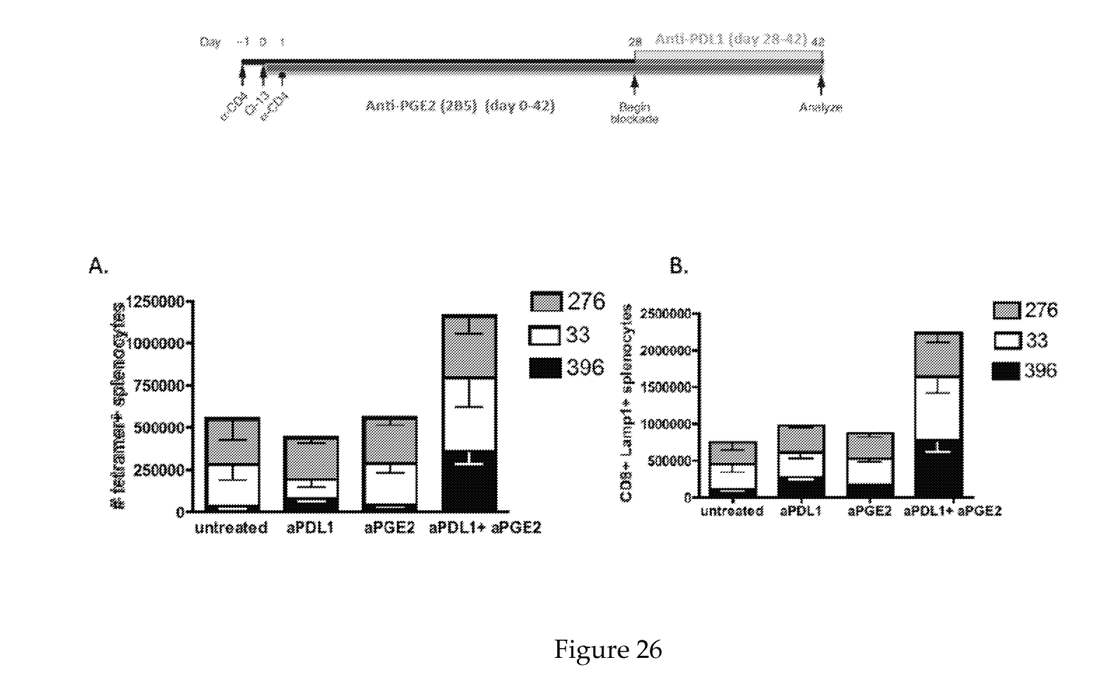

A beneficial effect on the antiviral CTL response during chronic LCMV infection was observed when mice are treated with either EP2/4 antagonists or anti-PGE2 mAb in combination with anti-PDL1 at days 28-42 post-infection (FIGS. 23 and 25). A greater beneficial effect was observed when PGE2 signaling was blocked with either EP2/4 antagonists or anti-PGE2 mAb at the beginning of infection, and then blocked later with anti-PDL1 mAb (i.e., beginning at day 28) (FIGS. 24 and 26).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing CTL exhaustion in a subject in need thereof, said method comprising administering a therapeutically effective amount of at least one inhibitor to the subject, wherein after the at least one inhibitor is administered to the subject, the CTL exhaustion is reduced.

2. The method of claim 1, wherein the at least one inhibitor diminishes the level of at least one selected from the group consisting of EP2, EP4, and PGE2.

3. The method of claim 1, wherein the at least one inhibitor diminishes the level of at least one selected from the group consisting of PD-1, PD-L1, and PD-L2.

4. The method of claim 1, wherein the at least one inhibitor is at least one selected from the group consisting of a chemical compound, a protein, a peptide, a peptidomemetic, an antibody, a ribozyme, a small molecule chemical compound, and an antisense nucleic acid molecule.

5. The method of claim 1, wherein the at least one inhibitor is at least one selected from the group consisting of AH6809, L161,982, PF-04418948, CJ-23423, an anti-PGE2 antibody and anti-PD-L1 antibody.

6. The method of claim 1, wherein the subject has at least one selected from the group consisting of a viral infection and a cancer.

7-14. (canceled)

15. A method of treating or preventing CTL exhaustion in a subject in need thereof, said method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the CTL exhaustion is reduced.

16. The method of claim 15, wherein the first of the at least two inhibitors is at least one selected from the group consisting of a chemical compound, a protein, a peptide, a peptidomemetic, an antibody, a ribozyme, a small molecule chemical compound, and an antisense nucleic acid molecule.

17. The method of claim 15, wherein the second of the at least two inhibitors is at least one selected from the group consisting of a chemical compound, a protein, a peptide, a peptidomemetic, an antibody, a ribozyme, a small molecule chemical compound, and an antisense nucleic acid molecule.

18. The method of claim 15, wherein the first of the at least two inhibitors is at least one selected from the group consisting of AH6809, L161,982, PF-04418948, CJ-23423, an anti-EP2 antibody, and anti-EP4 antibody and an anti-PGE2 antibody.

19. The method of claim 15, wherein the second of the at least two inhibitors is at least one selected from the group consisting of an anti-PD-1 antibody, and anti-PDL-1 antibody and an anti-PDL-2 antibody.

20. The method of claim 15, wherein the subject has at least one selected from the group consisting of a viral infection and a cancer.

21-28. (canceled)

29. A method of treating a viral infection in a subject in need thereof, said method comprising administering a therapeutically effective amount of at least one inhibitor to the subject, wherein after the at least one inhibitor is administered to the subject, the viral infection is treated.

30. The method of claim 29, wherein the at least one inhibitor diminishes the level of at least one selected from the group consisting of EP2, EP4, and PGE2.

31. The method of claim 29, wherein the at least one inhibitor diminishes the level of at least one selected from the group consisting of PD-1, PD-L1, and PD-L2.

32. The method of claim 29, wherein the at least one inhibitor is at least one selected from the group consisting of a chemical compound, a protein, a peptide, a peptidomemetic, an antibody, a ribozyme, a small molecule chemical compound, and an antisense nucleic acid molecule.

33. The method of claim 29, wherein the at least one inhibitor is at least one selected from the group consisting of AH6809, L161,982, PF-04418948, CJ-23423, an anti-PGE2 antibody and anti-PD-L1 antibody.

34. The method of claim 29, wherein the viral infection is a chronic viral infection.

35. The method of claim 29, wherein the viral infection is at least one selected from the group consisting of human immunodeficiency virus (HIV), hepatitis B virus (HBV), hepatitis C virus (HCV), Epstein-Barr virus (EBV), cytomegalovirus (CMV), varicella zoster virus (VZV) and herpes simplex virus (HSV).

36. (canceled)

37. A method of treating cancer in a subject in need thereof, said method comprising administering a therapeutically effective amount of at least one inhibitor to the subject, wherein after the at least one inhibitor is administered to the subject, the cancer is treated.

38. The method of claim 37, wherein the at least one inhibitor diminishes the level of at least one selected from the group consisting of EP2, EP4, and PGE2.

39. The method of claim 37, wherein the at least one inhibitor diminishes the level of at least one selected from the group consisting of PD-1, PD-L1, and PD-L2.

40. The method of claim 37, wherein the at least one inhibitor is at least one selected from the group consisting of a chemical compound, a protein, a peptide, a peptidomemetic, an antibody, a ribozyme, a small molecule chemical compound, and an antisense nucleic acid molecule.

41. The method of claim 37, wherein the at least one inhibitor is at least one selected from the group consisting of AH6809, L161,982, PF-04418948, CJ-23423, an anti-PGE2 antibody and anti-PD-L1 antibody.

42. (canceled)

43. A method of treating a viral infection in a subject in need thereof, said method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the viral infection is treated.

44. The method of claim 43, wherein the first of the at least two inhibitors is at least one selected from the group consisting of a chemical compound, a protein, a peptide, a peptidomemetic, an antibody, a ribozyme, a small molecule chemical compound, and an antisense nucleic acid molecule.

45. The method of claim 43, wherein the second of the at least two inhibitors is at least one selected from the group consisting of a chemical compound, a protein, a peptide, a peptidomemetic, an antibody, a ribozyme, a small molecule chemical compound, and an antisense nucleic acid molecule.

46. The method of claim 43, wherein the first of the at least two inhibitors is at least one selected from the group consisting of AH6809, L161,982, PF-04418948, CJ-23423, an anti-EP2 antibody, and anti-EP4 antibody and an anti-PGE2 antibody.

47. The method of claim 43, wherein the second of the at least two inhibitors is at least one selected from the group consisting of an anti-PD-1 antibody, and anti-PDL-1 antibody and an anti-PDL-2 antibody.

48. The method of claim 43, wherein the viral infection is a chronic viral infection.

49. The method of claim 43, wherein the viral infection is at least one selected from the group consisting of human immunodeficiency virus (HIV), hepatitis B virus (HBV), hepatitis C virus (HCV), Epstein-Barr virus (EBV), cytomegalovirus (CMV), varicella zoster virus (VZV) and herpes simplex virus (HSV).

50. (canceled)

51. A method of treating cancer in a subject in need thereof, said method comprising administering a therapeutically effective amount of at least two inhibitors to the subject, wherein the first of the at least two inhibitors inhibits at least one of EP2, EP4 and PGE2, and wherein the second of the at least two inhibitors inhibits at least one of PDL-1, PDL-2 and PD-1, and wherein after the at least two inhibitors are administered to the subject, the cancer is treated.

52. The method of claim 51, wherein the first of the at least two inhibitors is at least one selected from the group consisting of a chemical compound, a protein, a peptide, a peptidomemetic, an antibody, a ribozyme, a small molecule chemical compound, and an antisense nucleic acid molecule.

53. The method of claim 51, wherein the second of the at least two inhibitors is at least one selected from the group consisting of a chemical compound, a protein, a peptide, a peptidomemetic, an antibody, a ribozyme, a small molecule chemical compound, and an antisense nucleic acid molecule.

54. The method of claim 51, wherein the first of the at least two inhibitors is at least one selected from the group consisting of AH6809, L161,982, PF-04418948, CJ-23423, an anti-EP2 antibody, and anti-EP4 antibody and an anti-PGE2 antibody.

55. The method of claim 51, wherein the second of the at least two inhibitors is at least one selected from the group consisting of an anti-PD-1 antibody, and anti-PDL-1 antibody and an anti-PDL-2 antibody.

56-61. (canceled)


SEQUENCE LISTING (10 sequences)

SEQ ID NO: 1 - DNA, 18 bp, Mus musculus
ccggggttctggggaatc

SEQ ID NO: 2 - DNA, 21 bp, Mus musculus
gtgcatgcgaatgaggttgag

SEQ ID NO: 3 - DNA, 24 bp, Mus musculus
ttgccaagttctaattccatcaga

SEQ ID NO: 4 - DNA, 21 bp, Mus musculus
gtgcatgcgaatgaggttgag

SEQ ID NO: 5 - DNA, 24 bp, Mus musculus
gttagatggggggaggggacaact

SEQ ID NO: 6 - DNA, 23 bp, Mus musculus
tctgtgaagcgagtccttaggct

SEQ ID NO: 7 - DNA, 27 bp, Mus musculus
ggaaaacctcccggacttggttttcag

SEQ ID NO: 8 - DNA, 27 bp, Mus musculus
atcgccttctatcgccttcttgacgag

SEQ ID NO: 9 - DNA, 27 bp, Mus musculus
ggaaaacctcccggacttggttttcag

SEQ ID NO: 10 - DNA, 27 bp, Mus musculus
cagtattacaggagtgacccagatgtg